(19) United States
(12) Patent Application Publication
(10) Pub. No.: US 2010/0044229 A1
(43) Pub. Date: Feb. 25, 2010

(54) Electrophoretically Enhanced Detection of Analytes on a Solid Support

(75) Inventors: Ilana MARGALIT, Ramat Gan (IL); Galia Feldman, Ashdod (IL); Svetlana Lifshits, Rehovot (IL)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/501,366

(22) Filed: Jul. 10, 2009

Related U.S. Application Data
(60) Provisional application No. 61/160,097, filed on Mar. 13, 2009.
(60) Provisional application No. 61/083,211, filed on Jul. 24, 2008.
(60) Provisional application No. 61/080,087, filed on Jul. 11, 2008.

(51) Int. Cl. G01N 33/559

(52) U.S. Cl. 204/461; 204/606; 204/456

(57) ABSTRACT

The present embodiments provide systems, kits and methods suitable for performing dry or substantially dry electro-blotting analyses on immobilized protein or nucleic acid samples. Electro-blotting performed according to the presently described embodiments may include a step whereby detection of one or more immobilized proteins or nucleic acids is electrophoretically accelerated. Methods for performing electro-blotting of immobilized proteins or nucleic acids may include applying an electric voltage to one or more reagents typically used in protein or nucleic acid blotting procedure. The one or more reagents may be absorbed on a suitable carrier matrix. Electro-blotting performed in accordance with the systems and methods described herein may be performed under substantially dry conditions (i.e., with little or no aqueous buffers).

### CROSS-REFERENCE

This application claims the right of priority under 35 U.S.C. §19(e) to U.S. Provisional Application Ser. No. 61/160,097, filed Mar. 13, 2009, to U.S. Provisional Application Ser. No. 61/083,211, filed Jul. 24, 2008, and to U.S. Provisional Application Ser. No. 61/080,087, filed Jul. 11, 2008, all of which are commonly owned with the present application, and all of which are hereby expressly incorporated by reference in their entirety as though fully set forth herein.

### FIELD OF THE INVENTION

The present invention generally relates to the field of immunodetection/nucleic acid blotting, and more specifically to systems, kits and methods suitable for performing electro-immunodetection/electro-blotting of one or more immobilized analytes.

### BACKGROUND OF THE INVENTION

The separation and identification of proteins from biological samples is a key to understanding and learning to control the biochemistry of health and disease. One of the most widely used analytical techniques in the life sciences, Western blotting, or “immunoblotting”, is a post electrophoresis technique used for the detection and identification of antigens (such as e.g., proteins, nucleic acids, carbohydrates).

Immunodetection methods have advanced with the use of electro-blotting methods such as the electro blotting device and methods described by Margalit et al. (U.S. patent Appl. Publ. No. 2006/0272946. Using this dry-blotting system, proteins, nucleic acids, and other biomolecules are transferred from an electrophoretic separating gel to a blotting membrane much more efficiently and rapidly than traditional electro-blotting, and no liquid buffer handling is required by a user performing the electro-blotting method. For example, with this electro-blotting system an electro-blotting transfer can be performed in as little as 5 to 10 minutes.

Advances made in electrophoresis and blotting have pushed the limits of sample size and sensitivity with time now becoming a limiting factor of interest.

During a typical Western blotting procedure, an investigator will perform the following steps: (1) a protein sample will be prepared and subjected to sodium dodecyl sulfate polyacrylamide gel electrophoresis (SDS-PAGE): antigens present in the protein sample are resolved by relative mobility shift using electrophoresis; (2) the resolved proteins are transferred from the SDS-PAGE gel from Step (1) onto a solid support (e.g., nitrocellulose or PVDF membrane); (3) the membrane from Step (2) is incubated with a blocking reagent (typically a protein mixture such as non-fat milk, casein, bovine serum albumin, etc.) for about 1 hour to block any non-specific binding sites present on the membrane surface; (4) the blocked membrane is washed three times for 10 min each in a physiologically neutral buffer (e.g., PBS (phosphate buffered saline)) or PBST (PBS containing a small amount of a detergent, e.g., 0.1% Tween-20); (5) the membrane from Step (4) is incubated with a primary detection agent (e.g., and antibody) diluted in a solution for 1 hour to overnight. The primary detection agent binds the target antigen; (6) the membrane from Step (5) is removed from the primary detection agent solution and washed three times for 10 min each in PBS or PBST to remove the non-specifically bound primary detection agent; (7) the membrane from Step (6) is incubated with a secondary detection agent diluted in a solution for 1 hour. The secondary detection agent binds the primary detection agent. The secondary detection agent can be, but is not limited to, a detection agent linked (coupled) to a reporter enzyme such as alkaline phosphatase (AP) or horseradish peroxidase (HRP), which can be detected visually through the conversion of a calorimetric substrate (chromagen) to a colored precipitate at the site of a detection agent binding, (8) the membrane from Step (7) is removed from the secondary detection agent solution and washed three times for 10 min each in PBS or PBST to remove the non-specifically bound secondary detection agent; (9) a detection system such as luminescence or calorimetric system or other methods is used to detect the bound secondary detection agent. The duration of time, from Step (3) to (9) as described above, generally takes about 4.5 hours (seven steps).

In a typical conventional Western blot, the steps from Step (3) to Step (9) are performed on an orbital shaker or rocker. A typical conventional Western blot involves three incubation steps: one is the incubation with the blocking solution, the second is between the membrane and the primary detection agent; and another one is the incubation between the membrane and the secondary detection agent. Each incubation step usually takes about one hour.

The detection of nucleic acid hybridization events is a fundamental measurement in a variety of different life science research, diagnostic, forensic and related applications. A common feature of nucleic acid hybridization assays is that target and probe nucleic acids are combined under hybridization conditions and any hybridization events occurring between complementary target and probe nucleic acids are detected. The detection of hybridization events, i.e. target/probe duplexes, is then used to derive information about the source of the target nucleic acids, e.g. the genes expressed in a cell or tissue type, and the like.

In currently employed hybridization assays, the target nucleic acid must be labeled with a detectable label (where the label may be either directly or indirectly detectable), such that the presence of probe/target duplexes can be detected following hybridization. Currently employed labels include isotopic and fluorescent labels, where fluorescent labels are gaining in popularity as the label of choice, particularly for array based hybridization assays.

Currently, hybridization assays (such as, e.g. Southern blots and northern blots) are time consuming and require several hours or up to a day, as well as multiple changes in hybridization and washing buffer.

There exists a need for a system than can perform analyte detection assays in less than one hour and minimizing the number of steps required to perform the assay.

### SUMMARY OF THE INVENTION

The presently described embodiments provide systems, kits and methods suitable for performing dry or substantially dry electro-detection or electro-nucleic acid detection (hereinafter referred to as electro-blotting) analyses on immobilized protein or nucleic acid samples. Electro-blotting experiments performed according to the presently described embodiments may include a step whereby detection of one or more immobilized proteins or nucleic acids is electrophoretically accelerated. Methods for performing electro-blotting of immobilized proteins or nucleic acids may include applying an electric voltage to one or more reagents typically used in a blotting or nucleic acid blotting procedure. In some embodiments, certain of the reagents required to perform a protein or nucleic acid blotting experiment may be absorbed on a suitable carrier matrix. Electro-blotting performed in accordance with the systems and methods described herein may be performed under substantially dry conditions (i.e., with little or no aqueous buffers). Typically, dry or substantially dry electro-blotting procedures may be performed with 20 ml or less of an aqueous buffer, with 15 ml or less of an aqueous buffer, with 10 ml or less of an aqueous buffer, with 5 ml or less of an aqueous buffer, and most with 3 ml or less of an aqueous buffer, or with 1 ml or less of an aqueous buffer. The aqueous buffers may include diluents for diluting blocking reagents, hybridization reagents, primary antibodies, secondary antibodies, nucleic acid probes (RNA/DNA/PNA and the like), as well as any wash buffers required for further processing.

In some embodiments, electro-blotting performed in accordance with the systems, methods and kits described herein may be accomplished in less than 30 minutes. In some embodiments, electro-blotting performed in accordance with the systems, methods and kits described herein may be accomplished in less than 20 minutes. In some embodiments, electro-blotting performed in accordance with the systems, methods and kits described herein may be accomplished in less than 15 minutes. In some embodiments, electro-blotting performed in accordance with the systems, methods and kits described herein may be accomplished in less than 5 minutes. In some embodiments, electro-blotting performed in accordance with the systems, methods and kits described herein may be accomplished in less than 3 minutes.

In one embodiment, an electro-blotting system may include a first gel matrix body, a second gel matrix body and one or more carrier matrices. The first and second gel matrix bodies may include gel matrix ion reservoirs and can be provided to a customer in pre-made, disposable forms for use in a electro-blotting system. The pre-made, disposable first and second gel matrix bodies can be enclosed within a sealed package. Furthermore, multiple anodic and/or cathodic gel matrix ion reservoirs can be enclosed together in packaging. In some embodiments, one or more of the first and second gel matrix bodies may be supplied with a disposable tray for ease of handling. The tray may be a plastic tray or any other suitable material.

A carrier matrix may be made of a material that exhibits rapid absorption of liquids or aqueous solutions having macromolecules (e.g., polypeptide, antibody, nucleic acids, and the like) dispersed or absorbed therein, but which freely releases such macromolecules under the appropriate conditions while minimizing the irreversible absorption or coupling of such macromolecules to the carrier matrix. Materials suitable for use as carrier matrices in accordance with the embodiments described herein include any materials that release between 45% to about 95% or more of a biomolecular sample present in an electro-blotting mixture absorbed on the carrier matrix within 10 minutes when an electric current of at least 3 volts is applied across the carrier matrix.

In some embodiments, a carrier matrix having a substantially smooth surface may be selected so that the appearance of “pixelated of bands” (i.e., graininess) in experimental results may be minimized. Exemplary carrier matrices may include, though are not limited to, polyester fibers, polycarbonate fibers, hydrophilic cellulose fibers, cellulose acetate fibers, hydroxylated polyamide fibers (e.g., LOPRODYNE®), polyethersulfone fibers, acrylic co-polymer fibers, mixed cellulose ester fibers, modified poly(tetrafluoroethene) (PTFE), filter paper, felt, or combinations thereof. In some embodiments, a carrier matrix may include one or more sheets of blotting paper. In an embodiment, a carrier matrix may include one or more sheets of filter paper. In an embodiment, a carrier matrix may include one or more sheets of synthetic microfibers. Synthetic microfibers used in such sheets may include polyester microfibers, polyamide microfibers, or a combination of both polyester and polyamide microfibers. In some embodiments, a carrier matrix may include one or more microfiber sheets having between about 10% to about 90% polyester microfibers. In some embodiments, a carrier matrix may include one or more microfiber sheets having between about 10% to about 90% polyamide microfibers. In some embodiments, a carrier matrix may include one or more composite microfiber sheets having between about 10% to about 90% polyester microfibers in combination with between about 10% to about 90% polyamide microfibers. In some embodiments, a carrier matrix may include one or more microfiber sheets having about 80% polyester and about 20% polyamide microfibers.

Also provided for herein, in another aspect, are electrode assemblies for performing electro-blotting, in which the electrode assemblies include a body of gel matrix that includes a source of ions; and an electrically conducting electrode associated with the body of gel matrix. In certain embodiments, the electrode is attached to the body of gel matrix. In certain embodiments, the electrically conducting electrode is at least partially embedded in the body of gel matrix. In certain embodiments, the body of gel matrix is juxtaposed with the conducting electrode in a plastic tray before and during electrophoretic transfer. The electrode assembly can be enclosed in a sealed package. An electrode used in the dry electro-blotting systems and electrode assemblies provided herein can be, for example, a layer that includes a non-metallic electrically conducting material, a mesh comprising a non-metallic electrically conducting material, a metal foil, a metal mesh, non-conducting polymer coated with a conducting metal or nonmetal, and/or combinations thereof. An electrode of a non-conducting material coated with a conducting material can be in the form of a sheet, mesh, or other structure. In certain embodiments, an electrode of an electrode assembly comprises an electrochemically ionizable metal such as lead, copper, silver or combinations thereof. In certain embodiments, an electrode of an electrode assembly comprises aluminum or palladium.

In an embodiment, an electro-blotting system may include an anodic assembly, a cathodic assembly and a carrier matrix positionable therebetween. In an embodiment, the carrier matrix may be positioned between the anodic and the cathodic assemblies. An anodic assembly may include an anodic gel matrix body and an anodic electrode coupled thereto. A cathodic assembly may include a cathodic gel matrix body and a cathodic electrode coupled thereto.

An electrode assembly having an electrode in association with a gel matrix body may also be provided in a pre-made, disposable form, thereby facilitating use of the electrode assembly, and providing an effective business model. The electrode may be juxtaposed with a body of gel matrix, and may be provided in a tray or holder. The electrode assembly may be enclosed in a sealed package.

In a further embodiment, a dry electro-blotting system is provided, in which the system includes a blotting stack that includes a carrier matrix, a blotting membrane, an anode, a body of anodic gel matrix in contact with the anode and positioned between the anode and the blotting stack, a cathode, and a body of cathodic gel matrix in contact with the cathode and positioned between the cathode and the blotting stack.

In an embodiment, an anodic gel matrix and a cathodic gel matrix each include a source of ions suitable for electrophoresis. The electro-blotting system does not require the addition of substantial amounts of liquid buffers to the system before or during electro-blotting (such as when the blotting stack is being assembled). In some embodiments, the system may be assembled such that the anodic gel matrix and anode are on the membrane side of the blotting stack, and the cathodic gel matrix and cathode are on the carrier matrix side of the blotting stack. In some embodiments, the anode, the cathode, or both may be integral to a housing. In some embodiments, the anode, the cathode, or both may be integral to and/or coupled to a power supply. In some embodiments, the anode, the cathode, or both can be separate from a power supply.

In some embodiments, an electro-blotting system may include an apparatus for blotting antigens coupled to a solid support. An apparatus in accordance with such embodiments may include: a power supply that can hold a blotting stack, an anode, a body of anodic gel matrix juxtaposed with the anode between the anode and the blotting stack, a cathode, and a body of cathodic gel matrix juxtaposed with the cathode between the cathode and the blotting stack, during electro-blotting. During electro-blotting, the dry or substantially dry electro-blotting apparatus does not include, hold, or connect to any reservoirs for holding liquid buffers for electrophoresis. In some embodiments, the anode and anodic gel matrix of the apparatus are provided as an anode assembly that can be reversibly positioned on or against or connected with electrical contacts of the apparatus. In some embodiments, one or both of the anode or cathode is integral to the apparatus.

In an embodiment, an anodic electrode may be made of copper. In certain illustrative embodiments, both the anodic and cathodic electrodes may be made of copper.

In an embodiment, an electro-blotting system may optionally include a second carrier matrix. The second carrier matrix may be substantially the same as a first carrier matrix. The second carrier matrix may be made of a different material than that of the first carrier matrix. In an embodiment, a first carrier matrix and a second carrier matrix may be used simultaneously when performing an electro-blotting procedure. In an alternate embodiment, a second carrier matrix may be used sequentially to a first carrier matrix.

In an embodiment, an electro-blotting kit may include, in at least a first suitable container, one or more anodic assemblies, each including an anodic gel matrix body and an anodic electrode coupled thereto, one or more cathodic assemblies, each including a cathodic gel matrix body and a cathodic electrode coupled thereto, one or more first carrier matrices, and optionally one or more second carrier matrices. One or more of the gel matrix bodies may be configured to be positioned in or on a plastic tray supplied with the kit. Such a tray may facilitate handling of the gel matrix bodies during use.

In one embodiment, a kit for performing dry or substantially dry electro-blotting may include at least one body of gel matrix that comprises an ion source for electrophoresis and at least one suitable carrier matrix. In another embodiment, a kit includes at least one body of anodic gel matrix and at least one body of cathodic gel matrix.

A body of anodic gel matrix and a body of cathodic gel matrix may be provided in a kit in sealed packages. Electro-blotting gel matrix kits as presently contemplated may optionally include at least one blotting membrane, at least one sheet of filter paper, at least one sponge, and/or at least one electrode. The carrier matrix may be supplied in a separate package from the anodic and cathodic gel matrices. The carrier matrix may be packaged either alone, or packaged with a plurality of other carrier matrices.

A kit may further include one or more bottles of an appropriate diluent. Exemplary diluents include, by way of non-limiting example, phosphate buffered saline (PBS), Tris-buffered saline (TBS), Hank's buffer, Tris-EDTA (TE), Tris-EDTA-NaCl (TEN) or WESTERN BREEZE™ diluent, synthetic blocking buffer from BioFX™ or the like. The diluent may optionally include protease inhibitors, proteins, detergents, preservatives, antimicrobial agents or any combinations thereof.

A kit may further include one or more reagents necessary for performing blotting procedures. Non-limiting examples of such additional reagents include primary antibodies, loading control antibodies, secondary antibodies, blocking reagents and developing reagents (such as, e.g., chromogenic developing agents or chemiluminescent developing agents).

In an embodiment, a kit may include one or more disposable anodic electrode assemblies and/or one or more disposable cathodic electrode assemblies. In some embodiments, one or more anodic electrode assemblies can include a body of gel including a source of ions and an electrode juxtaposed with a body of gel matrix. In some embodiments, one or more cathodic electrode assemblies can include a body of gel including a source of ions and an electrode juxtaposed with a gel matrix. An anodic electrode assembly, a cathodic assembly, or both, can be provided in a tray, such as a plastic tray. An anodic assembly or a cathodic assembly can be provided in a tray, such as a disposable plastic tray.

The anodic and/or cathodic electrode assemblies can be enclosed within a sealed package together, or separately. Furthermore, multiple anodic and/or cathodic electrode assemblies can be enclosed together in packaging. In some aspects, an electro-blotting kit may include one or more disposable anodic electrode assemblies and one or more disposable cathodic electrode assemblies. In some aspects, an electro-blotting kit includes one or more disposable anodic electrode assemblies and at least one body of cathodic gel matrix. The kits may optionally include one or more blotting membranes, sheets of filter paper, sponges or carrier matrices.

In an embodiment, a method for performing electro-blotting on a protein sample may include providing an anodic assembly and a cathodic assembly. An anodic assembly may include an anode and a source of ions for electrophoresis. A cathodic assembly may include a cathode and a source of ions for electrophoresis. In one embodiment, a source of ions may be in the form of a gel matrix. The gel matrix may be electrically coupled to an anode or cathode. The anodic and cathodic assemblies may be coupled to electrical power supply such that an electric voltage may be passed therebetween.

A method for performing electro-blotting may further include providing a carrier matrix and contacting the carrier matrix with a proteinaceous or hybridization composition. A carrier matrix may be made of a material that exhibits rapid absorption of liquids or aqueous solutions having macromolecules (e.g., polypeptide, antibodies, nucleic acids, and the like) dispersed or absorbed therein, but which freely releases such macromolecules under the appropriate conditions while minimizing the irreversible absorption or coupling of such macromolecules to the carrier matrix. Materials suitable for use as carrier matrices in accordance with the embodiments described herein include materials that release at least 75% or more, at least 80% or more, at least 85% or more, at least 90% or more, or at least 95% or more of proteins present in a protein mixture absorbed on the carrier matrix. In some embodiments, a carrier matrix having substantially smooth surface may be selected so that the appearance of “pixelated bands” (i.e., graininess) in experimental results may be minimized. Exemplary carrier matrices may include, though are not limited to, polyester fibers, polycarbonate fibers, hydrophilic cellulose fibers, cellulose acetate fibers, hydroxylated polyamide fibers (e.g., LOPRODYNE®), polyethersulfone fibers, acrylic co-polymer fibers, mixed cellulose ester fibers, modified poly(tetrafluoroethene) (PTFE), filter paper, felt, or combinations thereof. In some embodiments, a carrier matrix may include one or more sheets of blotting paper. In an embodiment, a carrier matrix may include one or more sheets of synthetic microfibers. Synthetic microfibers used in such sheets may include polyester/polyamide microfibers as described previously. In an embodiment, a carrier matrix may include one or more sheets on filter paper.

In an embodiment, contacting a proteinaceous or hybridization composition with a carrier matrix may include preparing a buffered aqueous solution having one or more proteins or nucleic acids dissolved or dispersed therein. In an embodiment, a proteinaceous or hybridization composition may include at least one blocking reagent. The blocking agent may be a protein blocking agent or a nucleic acid blocking agent.

In an embodiment, a proteinaceous or hybridization composition may include at least one primary antibody. The primary antibody may be a loading control antibody. The primary antibody may be a user-defined antibody. The primary antibody may be provided in a kit or may be supplied by the end-user. In an embodiment, a proteinaceous or hybridization composition may include at least one secondary antibody. The secondary antibody may be coupled to horseradish peroxidase, biotin, alkaline phosphatase, a fluorescent dye or Qdot nanocrystals.

In an embodiment, a proteinaceous or hybridization composition may include at least one nucleic acid probe. The nucleic acid probe may be labeled or unlabeled. The nucleic acid probe may be DNA, RNA or PNA. The nucleic acid probe may be a synthetic oligonucleotide, or may be isolated from a naturally occurring or recombinant source. In an embodiment, a proteinaceous or hybridization composition may include at least one blocking reagent in combination with at least one primary antibody. In another embodiment, a proteinaceous or hybridization composition may include at least one blocking reagent in combination with at least one secondary antibody. In yet another embodiment, a proteinaceous or hybridization composition may include at least one blocking reagent in combination with at least one primary antibody and at least one secondary antibody.

A method of performing electro-blotting may include obtaining a protein blotting membrane having one or more protein or nucleic acid samples coupled to one surface thereof. The blotting membrane may be positioned on the anodic assembly such that the surface of the membrane lacking the protein or nucleic acid sample is substantially juxtaposed with and electrically coupled to the anodic gel matrix body thereof, and the surface of the membrane having the protein sample coupled thereto faces upward. The anodic assembly may be positioned in a disposable plastic tray.

A method of performing electro-blotting may further include preparing a proteinaceous or hybridization composition and absorbing at least a portion thereof to the carrier matrix. The proteinaceous or hybridization composition may include an appropriate blocking reagent, a primary antibody, a secondary antibody, a nucleic acid probe, or any combinations thereof, dispersed in an appropriate aqueous buffer. The carrier matrix with the absorbed proteinaceous or hybridization composition may be positioned over the protein blotting membrane such that it is substantially juxtaposed with the protein sample coupled to a surface of the blotting membrane.

In an embodiment, a second carrier matrix having a proteinaceous or hybridization composition absorbed thereon may be positioned on top of the first carrier matrix such that the second carrier matrix is substantially juxtaposed therewith. The proteinaceous or hybridization composition may include an appropriate blocking reagent, a primary antibody, a secondary antibody, a nucleic acid probe, or any combinations thereof, dispersed in an appropriate aqueous buffer. In an alternate embodiment, the first carrier matrix may be replaced with the second carrier matrix after at least one of an electro-blotting procedure has been performed, as described below.

In an embodiment, a method of performing an electro-blotting procedure may include positioning the cathodic assembly over the first and second carrier matrices such that the gel matrix body of the cathode is substantially juxtaposed and in electrical communication therewith, and the cathode is available for coupling to one or more additional components such as, e.g., a housing. It should be noted that any of the above described steps may include an optional de-bubbling step such that any air pockets formed between any of the assembled components are thereby removed.

In an embodiment, the assembly described above may be placed in an appropriate housing that is electrically coupled to a source of AC/DC power, which is configured to apply pressure to the assembled components and to facilitate the passage of an electric current therethrough.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The above brief description as well as further objects, features and advantages of the methods and apparatus of the present invention will be more fully appreciated by reference to the following detailed description of presently preferred but nonetheless illustrative embodiments in accordance with the present invention when taken in conjunction with the accompanying drawings:

FIG. 1A is a depiction of an electro-immunodetection system in accordance with an embodiment;

FIG. 1B is a depiction of an electro-immunodetection system in accordance with a further embodiment;

FIG. 1C is a depiction of an electro-immunodetection system in accordance with yet a further embodiment;

FIG. 2A is a flowchart depicting a method for performing an electro-blotting procedure in accordance with an embodiment;

FIG. 2B is a flowchart depicting a method for performing an electro-blotting procedure in accordance with an alternate embodiment;

FIG. 3 is an image demonstrating the inherent negative charge at neutral pH of various reagents used with an electro-immunodetection system according to an embodiment. Samples were resolved on a native 1.2% E-GEL® clear and the gel was stained with Coomassie to visualize resolved proteins. Samples are as follows: lane 1, WESTERNBREEZE® Blocking Solution; lane 2, mouse anti-actin monoclonal antibody; lane 3, mouse anti-tubulin monoclonal antibody; lane 4, goat anti-rabbit secondary antibody coupled to alkaline phosphatase; lane 5, goat anti-mouse secondary antibody coupled to alkaline phosphatase;

FIG. 4A shows results obtained after performing a blotting procedure to detect actin and tubulin in a SW480 whole cell lysate according to an embodiment;

FIG. 4B shows results obtained after performing a control blotting procedure to detect actin and tubulin in a SW480 whole cell lysate according to methods commonly used in the art;

FIG. 5A shows results obtained after performing a blotting procedure to detect actin and tubulin in a SW480 whole cell lysate according to an embodiment;

FIG. 5B shows results obtained after performing a blotting procedure to detect actin and tubulin in a SW480 whole cell lysate according to an alternate embodiment in which only pressure was applied to the system and without electrical current;

FIG. 6A shows results obtained after performing a blotting procedure to detect actin and tubulin in a SW480 whole cell lysate according to an embodiment, using filter paper as a carrier matrix according to an embodiment;

FIG. 6B shows results obtained after performing a blotting procedure to detect actin and tubulin in a SW480 whole cell lysate according to an embodiment, using a polyester/polyamide microfiber sheet as a carrier matrix according to an alternate embodiment;

FIG. 7A shows results obtained after performing a conventional blotting procedure to detect actin and tubulin in a SW480 whole cell lysate using the WESTERBREEZE™ protocol and the signal was detected using chemiluminescent methods (using HRP-conjugated secondary antibody and ECL reagents; upper panel) or chromogenic methods (using alkaline phosphatase-conjugated secondary antibody and WESTERNBREEZE™ reagents; lower panel);

FIG. 7B shows results obtained after performing an electro-blotting procedure to detect actin and tubulin in a SW480 whole cell lysate according to an embodiment, where blocking reagent as well as primary and secondary antibodies were applied to the carrier matrix prior to application of an electric voltage, and the signal was detected using chemiluminescent methods (using HRP-conjugated secondary antibody; upper panel) or chromogenic methods (using alkaline phosphatase-conjugated secondary antibody and WESTERNBREEZE™ reagents; lower panel);

FIG. 8A shows results obtained after performing a conventional blotting procedure to detect actin and tubulin in a SW480 whole cell lysate, where the protein sample was transferred to a nitrocellulose membrane using an IBLOT® apparatus, and the signal was detected using chemiluminescent methods (using an HRP-coupled secondary antibody and ECL detection; upper panel) or chromogenic methods (using an alkaline phosphatase-coupled secondary antibody and WESTERNBREEZE™ detection reagents; lower panel);

FIG. 8B shows results obtained after performing an electro-blotting procedure in accordance with an embodiment described herein to detect actin and tubulin in a SW480 whole cell lysate, where the protein sample was transferred to a nitrocellulose membrane using an IBLOT® apparatus, and the signal was detected using chemiluminescent methods (using an HRP-coupled secondary antibody and ECL detection; upper panel) or chromogenic methods (using an alkaline phosphatase-coupled secondary antibody and WESTERNBREEZE™ detection reagents; lower panel);

FIG. 9A shows results obtained after performing a conventional blotting procedure to detect actin and tubulin in a SW480 whole cell lysate, where the protein sample was transferred to a nitrocellulose membrane using conventional wet transfer methods, and the signal was detected using chemiluminescent methods (using an HRP-coupled secondary antibody and ECL detection; upper panel) or chromogenic methods (using an alkaline phosphatase-coupled secondary antibody and WESTERNBREEZE™ detection reagents; lower panel);

FIG. 9B shows results obtained after performing an electro-blotting procedure in accordance with an embodiment described herein to detect actin and tubulin in a SW480 whole cell lysate, where the protein sample was transferred to a nitrocellulose membrane using conventional wet transfer methods, and the signal was detected using chemiluminescent methods (using an HRP-coupled secondary antibody and ECL detection; upper panel) or chromogenic methods (using an alkaline phosphatase-coupled secondary antibody and WESTERNBREEZE™ detection reagents; lower panel);

FIG. 10A shows results obtained after performing a conventional blotting procedure to detect actin and tubulin in a SW480 whole cell lysate, where the protein sample was transferred to a PVDF membrane using an IBLOT® apparatus, and the signal was detected using chemiluminescent methods (using an HRP-coupled secondary antibody and ECL detection; upper panel) or chromogenic methods (using an alkaline phosphatase-coupled secondary antibody and WESTERNBREEZE™ detection reagents; lower panel);

FIG. 10B shows results obtained after performing an electro-blotting procedure in accordance with an embodiment described herein to detect actin and tubulin in a SW480 whole cell lysate, where the protein sample was transferred to a PVDF membrane using an IBLOT® apparatus, and the signal was detected using chemiluminescent methods (using an HRP-coupled secondary antibody and ECL detection; upper panel) or chromogenic methods (using an alkaline phosphatase-coupled secondary antibody and WESTERNBREEZE™ detection reagents; lower panel);

FIG. 11A shows results obtained after performing a conventional blotting procedure to detect actin and tubulin in a SW480 whole cell lysate, where the protein sample was transferred to a PVDF membrane using conventional wet transfer methods, and the signal was detected using chemiluminescent methods (using an HRP-coupled secondary antibody and ECL detection; upper panel) or chromogenic methods (using an alkaline phosphatase-coupled secondary antibody and WESTERNBREEZE™ detection reagents; lower panel);

FIG. 11B shows results obtained after performing an electro-blotting procedure in accordance with an embodiment described herein to detect actin and tubulin in a SW480 whole cell lysate, where the protein sample was transferred to a PVDF membrane using conventional wet transfer methods, and the signal was detected using chemiluminescent methods (using an HRP-coupled secondary antibody and ECL detection; upper panel) or chromogenic methods (using an alkaline phosphatase-coupled secondary antibody and WESTERNBREEZE™ detection reagents; lower panel);

FIG. 12A shows results obtained after performing a conventional blotting procedure to detect proteins in an E. coli cell lysate using the WESTERBREEZE™ protocol and the signal was detected using chemiluminescent methods using AP-conjugated secondary antibody and the WESTERBREEZE™ CL reagents;

FIG. 12B shows results obtained after performing a two-step blotting procedure according to an alternate embodiment to detect proteins in an E. coli cell lysate using the WESTERBREEZE™ protocol and the signal was detected using chemiluminescent methods using AP-conjugated secondary antibody and the WESTERBREEZE™ CL reagents;

FIG. 13A shows results obtained comparing conventional immunoblotting (left panel) with electro-immunoblotting (right panel) performed on A341 lysate in a single step. The indicated dilutions of primary (anti-EIF) and secondary antibody (monoclonal anti-mouse-HRP) were applied to a carrier matrix and electro-immunoblotting was performed in a single step using the indicated conditions;

FIG. 13B shows results obtained comparing conventional immunoblotting (left panel) with electro-immunoblotting (right panel) performed on HeLa cell lysate in a single step. The indicated dilutions of primary (anti-ERK) and secondary antibody (monoclonal anti-mouse-HRP) were applied to a carrier matrix and electro-immunoblotting was performed in a single step using the indicated conditions;

FIG. 14A shows results obtained comparing conventional immunoblotting (left panel) with electro-immunoblotting (right panel) performed on purified bovine serum albumin (BSA) in two sequential steps. The indicated dilution of primary antibody (anti-BSA) was applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 14B shows results obtained comparing conventional immunoblotting (left panel) with electro-immunoblotting (right panel) performed on SW480 cell lysate in two sequential steps. The indicated dilution of primary antibodies (anti-tubulin and anti-actin) were applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 14C shows results obtained comparing conventional immunoblotting (left panel) with electro-immunoblotting (right panel) performed on HeLa cell lysate in two sequential steps. The indicated dilution of primary antibody (anti-p70) was applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 14D shows results obtained comparing conventional immunoblotting (left panel) with electro-immunoblotting (right panel) performed on SW480 cell lysate in two sequential steps. The indicated dilution of primary antibody (anti-p53) was applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 15A shows results obtained comparing conventional immunoblotting (left panel) with electro-immunoblotting (right panel) performed on HeLa cell lysate after the electro-blotting protocol was optimized for the indicated antigen-antibody pairs. The indicated dilution of primary antibody (anti-4E-BP1) was applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 15B shows results obtained comparing conventional immunoblotting (left panel) with electro-immunoblotting (right panel) performed on SW480 cell lysate after the electro-blotting protocol was optimized for the indicated antigen-antibody pairs. The indicated dilution of primary antibody (anti-β-catenin) was applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 15C shows results obtained comparing conventional immunoblotting (left panel) with electro-immunoblotting (right panel) performed on rabbit HCG after the electro-blotting protocol was optimized for the indicated antigen-antibody pairs. The indicated dilution of primary antibody (rabbit anti-HCG) was applied to a carrier matrix and electro-immunoblotting was performed using the indicated conditions. Next, the indicated dilution of secondary antibody (monoclonal anti-rabbit-HRP) was applied to the carrier matrix and electro-immunoblotting was performed in a single step using the indicated conditions;

FIG. 15D shows results obtained comparing conventional immunoblotting (left panel) with electro-immunoblotting (right panel) performed on purified GST-tagged EGFR after the electro-blotting protocol was optimized for the indicated antigen-antibody pairs. The indicated dilution of primary antibody (anti-EGFR) was applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 15E shows results obtained comparing conventional immunoblotting (left panel) with electro-immunoblotting (right panel) performed on HeLa cell lysate after the electro-blotting protocol was optimized for the indicated antigen-antibody pairs. The indicated dilution of primary antibody (anti-IKK) was applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 16A shows results obtained comparing conventional immunoblotting (left panel) with electro-immunoblotting (right panel) performed in two sequential steps on cell lysate prepared from HeLa cells expressing recombinant His-tagged Src protein. The indicated dilution of primary antibody (anti-His) was applied to a carrier matrix and electro-blotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 16B shows results obtained comparing conventional immunoblotting (left panel) with electro-immunoblotting (right panel) performed in two sequential steps on recombinant Positope. The indicated dilution of primary antibody (anti-V5) was applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 16C shows results obtained comparing conventional immunoblotting (left panel) with electro-immunoblotting (right panel) performed in two sequential steps on recombinant Positope. The indicated dilution of primary antibody (anti-Myc) was applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 17A shows results obtained using SW480 cell lysate comparing conventional immunoblotting (left panel) with SNAP i.d. Protein Detection System (Millipore; center panel) and electro-immunoblotting in two sequential steps (right panel). SNAP i.d. was performed according to maufacturer's instruction using the indicated antibody dilutions. For electro-immunoblotting, the indicated dilution of primary antibody (anti-insulin) was applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 17B shows results obtained using purified GST-tagged EGFR comparing conventional immunoblotting (left panel) with SNAP i.d. Protein Detection System (Millipore; center panel) and electro-immunoblotting in two sequential steps (right panel). SNAP i.d. was performed according to manufacturer's instructions using the indicated antibody dilutions. For electro-immunoblotting, the indicated dilution of primary antibody (anti-EGFR) was applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 17C shows results obtained using purified SW480 lysate comparing conventional immunoblotting (left panel) with SNAP i.d. Protein Detection System (Millipore; center panel) and electro-immunoblotting in two sequential steps (right panel). SNAP i.d. was performed according to manufacturer's instructions using the indicated antibody dilutions. For electro-immunoblotting, the indicated dilution of primary antibodies (anti-tubulin and anti-actin) was applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions;

FIG. 17D shows results obtained using E. coli lysate comparing conventional immunoblotting (left panel) with SNAP i.d. Protein Detection System (Millipore; center panel) and electro-immunoblotting in two sequential steps (right panel). SNAP i.d. was performed according to manufacturer's instructions using the indicated antibody dilutions. For electro-immunoblotting, the indicated dilution of primary antibody (anti-E. coli) was applied to a carrier matrix and electro-immunoblotting was performed using the indicated conditions. Next, the indicated dilution of secondary antibody (monoclonal anti-mouse-HRP) was applied to the carrier matrix and electro-immunoblotting was performed in a single step using the indicated conditions.

FIG. 18A shows a control nucleic acid blotting experiment;

FIG. 18B shows an electro-blotting experiment using a labeled nucleic acid probe nucleic acid bound to a solid support in accordance with an embodiment;

FIG. 18C shows an electro-blotting experiment using a labeled nucleic acid probe nucleic acid bound to a solid support in accordance with an alternate embodiment;

While the invention is susceptible to various modifications and alternative forms, specific embodiments thereof are shown by way of example in the drawings and will herein be described in detail. The drawings may not be to scale. It should be understood that the drawings and detailed description thereto are not intended to limit the invention to the particular form disclosed, but to the contrary, the intention is to cover all modifications, equivalents and alternatives falling within the spirit and scope of the present invention as defined by the appended claims.

### DETAILED DESCRIPTION OF THE INVENTION

### Definitions

The terms used throughout this specification generally have their ordinary meanings in the art, within the context of the invention, and in the specific context where each term is used. Certain terms are discussed below, or elsewhere in the specification, to provide additional guidance to the practitioner in describing the devices and methods of the invention and how to make and use them. It will be appreciated that the same thing can be said in more than one way. Consequently, alternative language and synonyms may be used for any one or more of the terms discussed herein, nor is any special significance to be placed upon whether or not a term is elaborated or discussed in greater detail herein. Synonyms for certain terms are provided. A recital of one or more synonyms does not preclude the use of other synonyms. The use of examples anywhere in this specification, including examples of any terms discussed herein, is illustrative only, and in no way limits the scope and meaning of any of the embodiments set forth herein or of any exemplified term.

The term “immunoblot” as used herein is synonymous with the term “western blot”. Unless otherwise specified, for the purposes of the present disclosure, the two terms may be used interchangeably.

The terms “Southern blot”, is a method routinely used in molecular biology to check for the presence of a DNA sequence in a DNA sample. Southern blotting combines agarose gel electrophoresis for size separation of DNA with methods to transfer the size-separated DNA to a filter membrane for probe (typically nucleic acid) hybridization and subsequent detection.

The term “northern blot” refers to a process that is essentially identical to a Southern blot, except that the target molecule being detected is RNA rather than DNA. Accordingly, electrophoresis of the RNA sample that is to undergo northern blotting is typically, though not necessarily, carried out under denaturing conditions. The probe to which the target RNA molecule will hybridized is typically a nucleic acid (i.e., DNA, RNA or PNA) probe.

The term “western blot” and “immunoblot” may be used interchangeably and refer to is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

As used herein, the terms “gel matrix” and “gel matrix body” and the like generally refer to a discreet unit of a colloidal matrix, which colloid contains a source of ions, buffers and other constituents that make the body suitable for use in electrophoretic applications.

The term “substantially juxtaposed”, when used in the context of two surfaces being “substantially juxtaposed”, generally means that the two surfaces are in substantially continuous surface contact. In the context of the present application, the term means that at least 50% of the surfaces of the two juxtaposed objects are in continuous surface contact.

As used herein, the term “substantially dry” is meant to indicate that no additional reservoir of aqueous buffer is required to practice the presently described embodiments. It does not indicate an absence of liquids, but rather that the use of liquid buffers is minimized and that no vessel is required to hold any liquids. The use of liquids is, for example, contemplated to apply a detecting molecule such as, e.g., an antibody or a nucleic acid probe to a carrier matrix. Likewise, liquids are used to form the gel matrix stacks.

The terms “electro-blotting”, “electrically-enhanced blotting”, “electrically-assisted blotting” and the like, as used herein may be used interchangeably, and refer to the process of applying an electrical field to a detecting molecule (such as, e.g., a primary or secondary antibody, a nucleic acid probe, an oligonucleotide, an aptamer, an oligomer, a polypeptide or an oligopeptide, or labeled versions of any of the aforementioned) so that the detecting molecule is brought in proximity to a target analyte (i.e., a molecule that binds to the detecting molecule with a high degree of specificity). The term “electro-blotting” may refer to a subset of such embodiments, where either the detecting molecule, the target analyte, or both the detecting molecule and the target analyte are antibodies or antigen/antibody pairs.

### Electro-Blotting System:

The presently described embodiments provide for a substantially dry electro-blotting system, which system includes electro-blotting stack having one or more suitable carrier matrices positioned therein. The electro-blotting stack includes an anode, a body of anodic gel matrix, a cathode, and a body of cathodic gel matrix positioned between the cathode, in which the anodic gel matrix and the cathodic gel matrix each comprise an ion source for electrophoretic transfer. The electro-blotting stack further includes at least one carrier matrix positionable between the anodic gel matrix and the cathodic gel matrix. The carrier matrix may be in the form of a sheet.

An electro-blotting stack according to the present embodiments is configured to accept a protein or nucleic acid blotting membrane (or more simply “a blotting membrane”) positioned between the two gel body matrices. The blotting membrane may be any type of membrane used in the art for performing immuno- or nucleic acid blotting procedures. A wide variety of such membranes are know to the skilled artisan and may include, by way of non-limiting example, a nitrocellulose (NC) membrane, a nylon membrane, or a Polyvinylidene Fluoride (PVDF) membrane.

The blotting membrane may be supplied by the end user prior to use of the system. The blotting membrane will typically have one or more biomolecular samples (such as, for example, a polysaccharide, a protein, a peptide, or a nucleic acid) coupled to a surface of the blotting membrane. A biomolecular sample may be reversibly or irreversibly coupled to such a blotting membrane. Methods for coupling a biomolecular sample to a blotting membrane are widely know in the art and may include, without limitation, wet, semi-dry and dry electrophoretic transfer methods. Exemplary though non-limiting dry electrophoretic transfer methods are described in U.S. Patent Appl. Publ. Nos. 2006/0278531 and 20060272946 by Margalit et al., which applications are commonly owned with the present application and which are hereby expressly incorporated by reference in their entirety as though fully set forth herein. Other methods well known to one skilled in the art for coupling a biomolecular sample to a solid membrane support include, though are not limited to, dot blotting, spotting, vacuum transfer and capillary transfer.

In some embodiments, the electro-blotting system may be devoid of any extraneous buffers or of any reservoirs for holding or supplying liquid or aqueous buffers to the system during use. In this sense, the presently described electro-blotting system may be described as being “dry” or “substantially dry”. Such a statement is not intended to mean that the system is entirely devoid of liquids, but rather that no additional supply of buffer is required in order to practice various of the embodiments contemplated herein. For example, use of the term “dry” or “substantially dry” is not meant to imply that absorption of a blotting buffer to a carrier matrix as described herein may be achieved without use of an aqueous buffer. Nor is it meant to imply that, e.g., washing steps are to be performed without use of a buffer. Instead, the term is meant to convey that no extraneous source of liquid buffer or liquid buffer reservoir is required to supply ions used for electrophoresis to the system. On the contrary, in some embodiments one or more components of an electro-blotting system, such as the blotting membrane, the carrier matrix, or one or more sheet of filter paper placed between the layers of the blotting stack may be wetted prior to use of the system. In an electro-blotting system as presently contemplated however, wetting of one or more of the system components such as, e.g., a blotting membrane or sheet of filter paper with water, a detergent solution, an incubation buffer, a pre-hybridization buffer or other aqueous solution, is not necessary for providing ions required to drive electrophoretic transfer.

The system is constructed such that when an electrical current is passed between the cathode and the anode, molecules used during electro-blotting procedures (e.g., blocking reagents, primary antibodies, secondary antibodies, nucleic acid probes, and the like) that are absorbed on the carrier matrix are transferred from the carrier matrix to a blotting membrane juxtaposed therewith, where such molecules bind to the appropriate antigen present in the biomolecular sample coupled to a surface of the membrane.

The assembled system thus provides electrical continuity from the cathode to the anode, in which current passes from the cathode through the cathodic body of gel matrix, one or more carrier matrices, the blotting membrane, and the anodic body of gel matrix to the anode. Thus some embodiments, one side of the cathodic body of gel matrix is in contact with the cathode, and another side of the cathodic body of gel matrix is in direct or indirect electrical contact with a carrier matrix of the blotting stack. One side of the anodic body of gel matrix is in contact with the anode, and another side of the anodic body of gel matrix is in direct or indirect electrical contact with a blotting membrane of the stack.

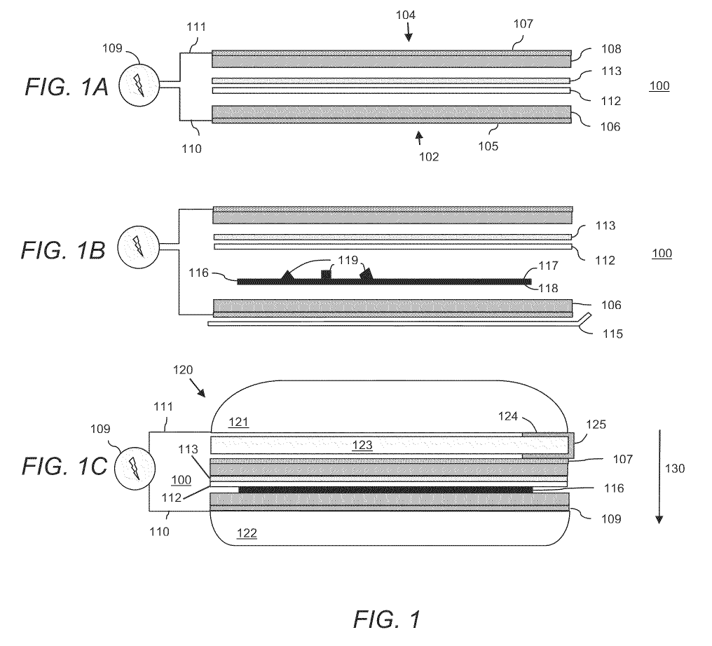

Turning now to FIG. 1, an electro-blotting system, including various components thereof, and their assembly and configuration prior to and during use according to certain embodiments will be discussed in detail. It will of course be readily apparent to one skilled in the art that additional components not discussed below may be included in various alternate embodiments of an electro-blotting system without departing from the spirit and scope thereof, so long as such additional components do not interfere with the functioning of the system as described below.

FIG. 1A depicts an electro-blotting stack according to an embodiment. Electro-blotting stack 100 may include lower stack 102 and upper stack 104. Lower stack 102 may also be referred to as anodic assembly 102. Likewise, upper stack 104 may also be referred to as cathodic assembly 104.

In an embodiment, lower stack 102 may include anode 105 and anodic gel matrix 106, and upper stack 104 may include cathode 107 and cathodic gel matrix 108. In an embodiment, anode 105 may be physically coupled to anodic gel matrix 106. Anode 105 may be electrically coupled to anodic gel matrix 106. In an embodiment, cathode 107 may be physically coupled to anodic gel matrix 108. Cathode 107 may be electrically coupled to cathodic gel matrix 108. Physical and electrical coupling of electrode to the gel matrix bodies are not mutually exclusive. In an embodiment, a surface of anode 105 may be juxtaposed with at least a portion of a surface of anodic gel matrix 106, as depicted in FIG. 1A. Likewise, a surface of cathode 107 may be juxtaposed with at least a portion of a surface of anodic gel matrix 108. In an alternate embodiment, lowerstack 102 may be manufactured such that at least a portion of anode 105 resides or is embedded in at least a portion of anodic gel matrix 106. Likewise, upper stack 104 may be manufactured such that at least a portion of cathode 107 resides or is embedded in at least a portion of cathodic gel matrix 108.

In an embodiment, the length and width of an anodic gel matrix body and a cathodic gel matrix may be selected such that both surfaces of a protein blotting membrane placed therebetween are in contact with at least one of the surfaces of the gel matrix bodies. Typically, the dimensions of the anodic gel matrix body and the cathodic gel matrix body will be substantially similar. Typically, the dimensions of the anodic gel matrix body and the cathodic gel matrix body will be substantially similar to the dimensions of electrodes coupled thereto. In an embodiment, the length of at least one side of an anodic gel matrix body and the length of at least one side of a cathodic gel matrix body will be in the range of about 2 cm to about 25 cm, about 5 cm to about 20 cm, about 8 cm to about 15 cm, or about 10 cm to about 12 cm. Likewise, the length of another side of an anodic gel matrix body and the length of another side of a cathodic gel matrix body will be in the range of about 2 cm to about 25 cm, about 5 cm to about 20 cm, about 8 cm to about 15 cm, or about 10 cm to about 12 cm. In an embodiment, each of the gel matrix bodies may have a thickness in the range of about 1 mm to about 15 mm, about 2 mm to about 10 mm, or about 3 mm to about 5 mm.

In an embodiment, the anode and the cathode may have substantially the same dimensions as the corresponding gel matrices. In an alternate embodiment, the electrodes may have substantially smaller dimensions as the corresponding gel matrix bodies.

### Gel Matrix Bodies:

A body of anodic gel matrix and a body of cathodic gel matrix of an electro-blotting system may have the same or different compositions. For example, a body of anodic gel matrix and a body of cathodic gel matrix of an electro-blotting system may have the same or different gel-forming polymers, or one or more common gel-forming polymers at different concentrations. A body of anodic gel matrix and a body of cathodic gel matrix of an electro-blotting system can have the same or different buffers, or can have a common buffer present at different concentrations. An anodic gel matrix may include one or more additional compounds not present in a cathodic gel matrix. A cathodic gel matrix may include one or more additional compounds not present in the anodic gel matrix.

A body of gel matrix (a body of anodic gel matrix or a body of cathodic gel matrix) may include agarose, acrylamide, alumina, silica, starch or other polysaccharides such as chitosan, gums (e.g., xantham gum, gellan gum), carrageenan, pectin, or other polymers that form gels, or any combinations of these. In some embodiments, a body of cathodic gel matrix may include acrylamide, for example, at a concentration of from about 2.5% to about 30%, or from about 5% to about 20%. In some embodiments, a body of cathodic gel matrix may include agarose, for example at a concentration of from about 0.1% to about 5%, or from about 0.5% to about 4%, or from about 1% to about 3%. In some embodiments, a body of cathodic gel matrix comprises acrylamide and agarose, for example, a cathodic gel matrix can comprise from about 2.5% to about 30% acrylamide and from about 0.1% to about 5% agarose, from about 5% to about 20% acrylamide and from about 0.2% to about 2.5% agarose.

A source of ions for electrophoretic transfer provided in a cathodic gel matrix or an anodic gel matrix may be from for example, a salt, acid, base, or buffer, or combinations thereof. In an embodiment, the body of cathodic gel matrix may include at least one buffer, such as an organic buffer. A buffer provided in the cathodic gel matrix may be a zwitterionic buffer. In certain embodiments in which the carrier matrix includes proteins or peptides to be electro-blotted, the body of cathodic gel matrix may include a buffer having a pKa of between about 6.5 and about 8.5, or between about 7 and about 8. A buffer in the cathodic gel matrix may be present at a concentration of from about 10 mM to about 1 M, for example, at a concentration of between about 20 mM and about 500 mM, a between about 50 mM and about 300 mM, or between about 60 mM and about 150 mM.

In an embodiment, the body of cathodic gel matrix may include, by way of nonlimiting example, 2-N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxy-propanesulfonic acid (MOPSO), N,N-bis-(hydroxyethyl)-2-aminoethanesulfonic acid (BES), 3-(N-morpholino)-propanesulfonic acid (MOPS), N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-Bis[2-hydroxyethyl]amino)-2-hydroxypropanesulfonic acid (DIPSO), N-(2-Hydroxyethyl)piperazine-N′-(2-hydroxypropanesulfonic acid) (HEPPSO), 4-(2-Hydroxyethyl)-1-piperazine propanesulfonic acid (EPPS) N-[Tris(hydroxymethyl)-methyl]glycine (Tricine), N,N-Bis(2-hydroxyethyl)glycine (Bicine), (2-Hydroxy-1,1-bis(hydroxymethyl)ethyl)amino]-1-propanesulfonic acid (TAPS), N-(1,1-Dimethyl-2-hydroxyethyl)-3-amino-2-hydroxypropanesulfonic acid (AMPSO), tris(hydroxy methyl)amino-methane (Tris), or bis[2-hydroxyethyl]iminotris-[hydroxymethyl]methane (BisTris).

The cathodic gel matrix, the anodic gel matrix, or both may optionally include an ion exchange matrix. For example, the anodic gel matrix may optionally include a cation exchange matrix. A cathodic gel matrix may optionally include an anion exchange matrix such as, by way of example, DEAE cellulose. The ion exchange matrix can be loaded with ions, such as buffer ions, for example, a DEAE ion exchange matrix can be loaded with Tricine anions.

A cathodic gel matrix body may further include ethylene glycol, an alcohol, one or more detergents, one or more anti-fungal agents or one or more anti-corrosion agents, etc.

A source of ions for electrophoretic transfer provided in the anodic gel matrix may be from a salt, acid, base, or buffer. In an embodiment, the body of anodic gel matrix may include at least one buffer, such as an organic buffer. A buffer provided in the anodic gel matrix may be a zwitterionic buffer. In certain embodiments in which a carrier matrix includes proteins or peptides to be electro-blotted, the body of anodic gel matrix may include a buffer having a pKa of between about 6 and about 8, or between about 6.2 and about 7.2. A buffer can be present at a concentration of from about 10 mM to about 1 M, for example, at a concentration of between about 20 mM and about 500 mM, between about 50 mM and about 300 mM, or between about 60 mM to about 150 mM.

In an embodiment, the body of anodic gel matrix may include 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxypropane-sulfonic acid (MOPSO), N,N-bis-(hydroxyethyl)-2-aminoethanesulfonic acid (BES), 3-(N-morpholino)-propanesulfonic acid (MOPS), N-tris-(hydroxymethyl)-2-ethanesulfonic acid (TES), N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-Bis[2-hydroxyethyl]amino)-2-hydroxypropanesulfonic acid (DIPSO), N-(2-Hydroxyethyl)-piperazine-N′-(2-hydroxypropanesulfonic acid) (HEPPSO), 4-(2-Hydroxyethyl)-1-piperazinepropanesulfonic acid (EPPS), N-[Tris(hydroxymethyl)methyl]-glycine (Tricine), N,N-Bis(2-hydroxyethyl)glycine (Bicine), (2-Hydroxy-1,1-bis-(hydroxymethyl)ethyl)amino]-1-propanesulfonic acid (TAPS), N-(1,1-Dimethyl-2-hydroxyethyl)-3-amino-2-hydroxypropanesulfonic acid (AMPSO), tris(hydroxy methyl)amino-methane (Tris), or bis[2-hydroxyethyl]iminotris[hydroxymethyl]methane (BisTris).

Without limiting the invention to any particular mechanism, it is contemplated that one or more species of anions present in an anodic gel matrix of an electro-blotting system that moves relatively fast when an electric field is established during electrophoretic transfer may, as it migrates rapidly to the anode, contribute to the electrophoretic concentration of migrating macromolecules which are absorbed on a carrier matrix as described below in greater detail, and which are also moving toward the anode, but are moving in a part of the field that lacks the fast-moving anions. In the context of electrophoretic movement, macromolecules that are migrating “behind” fast moving anions (that is, they are farther from the anode) may experience an electrophoretic concentration that is amplified by the depletion of the fast-moving ions from the anodic gel matrix as the fast-moving anions rapidly move to the anode.

The effect of anionic compounds provided exclusively in the anodic gel matrix also applies to anionic compounds that are present at a significantly reduced concentration in the cathodic gel matrix when compared with the anodic gel matrix. As used herein “significantly reduced concentration” means that the concentration of the anionic buffer compound in the cathodic matrix is 0.5× or less, 0.2× or less, or 0.1× or less when compared with the concentration of the anionic compound in the anodic gel matrix of an electro-blotting system or apparatus. Thus, in one embodiment, a cathodic stack and an anodic stack of an electro-blotting system may include the same anionic compound, in which the compound is present at different concentrations in the cathodic stack and the anodic stack.

Compounds provided in an anodic gel matrix of an electro-blotting apparatus, device or system that are not present, or present in significantly reduced amounts, in the cathodic gel matrix, may be buffer compounds that during electrophoretic transfer are present in the electro-blotting system in the form of anions, and are referred to herein as “anionic buffer compounds”. Anionic buffer compounds provided in the anodic gel matrix and not provided in the cathodic gel matrix (or provided in significantly reduced amount in the cathodic gel matrix) are “fast-moving” with respect to some other buffer compounds, including, for example, other anionic buffer compounds that may be provided in the cathodic gel matrix. Therefore the choice of anionic buffer compounds for preferential use in the anodic gel matrix will depend, in part, on the anionic compounds (such as buffers) provided in the cathodic gel matrix, the pH of the buffers in the anodic gel matrix and cathodic gel matrix, and the pKa's of the anionic buffer compounds. For example, anionic buffer compounds that may be provided in the anodic gel matrix of an electrophoretic transfer system in which electro-blotting occurs near neutral pH include compounds that have a pKa at or neutrality (between about pH 6 and about pH 8), in some examples between pH 6.0 and pH 8.0, and at least 0.5 log units below, such as, for example, about one log unit below, the pKa of one or more buffer compounds provided in the cathodic gel matrix.

In some embodiments, an anodic gel matrix of an electro-blotting system may include an anionic buffer compound that is not present in the cathodic gel matrix, in which the anionic compound has a pKa near or below neutrality and is present as an anion at or near neutral pH. In some embodiments, the compound may be a biological buffer having a pKa of less that about 7.5, or less than about 7.2, and in some embodiments below about 7.0, where the biological buffer compound forms an anion in solution during electrophoresis. In certain illustrative aspects, the anionic buffer has a pKₐ less than 7.5, 7.4, 7.3, 7.2, 7.1, 7.0, 6.9, 6.8, 6.7, 6.6, or 6.5.

Non-limiting examples of anionic compounds that may be present in an anodic gel matrix and not present in the cathodic gel matrix include EDTA, succinate, citrate, aspartic acid, glutamic acid, maleate, cacodylate, N-tris-(hydroxymethyl)-2-ethanesulfonic acid (TES), 2-(N-morpholino)-ethanesulfonic acid (MES), N-(2-Acetamido)iminodiacetic acid (ADA), N-(2-acetamido)-2-aminoethanesulfonic acid (ACES), piperazine-N,N′-2-ethanesulfonic acid (PIPES), 2-(N-morpholino)-2-hydroxypropanesulfonic acid (MOPSO), N,N-bis-(hydroxyethyl)-2-aminoethanesulfonic acid (BES), or 3-(N-morpholino)-propanesulfonic acid (MOPS). Such anionic buffer compounds can be used in electro-blotting systems in which the pKa of an anionic compound in the cathode compartment is greater than that of the anionic compound in the anode compartment. In these embodiments the cathode compartment of the system can include, for example, one or more of, glycine, N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-Bis[2-hydroxyethyl]amino)-2-hydroxypropanesulfonic acid (DIPSO), N-(2-Hydroxyethyl)piperazine-N′-(2-hydroxypropanesulfonic acid) (HEPPSO), 4-(2-Hydroxyethyl)-1-piperazine propanesulfonic acid (EPPS), N-[Tris(hydroxymethyl)methyl]glycine (Tricine), N,N-Bis(2-hydroxyethyl)glycine (Bicine), [(2-Hydroxy-1,1-bis(hydroxymethyl)ethyl)amino]-1-propanesulfonic acid (TAPS), and N-(1,1-Dimethyl-2-hydroxyethyl)-3-amino-2-hydroxypropanesulfonic acid (AMPSO).

In some embodiments, an anionic compound present in an anodic gel matrix that is not present (or is present at significantly reduced concentration) in a cathodic gel matrix is a zwitterionic buffer with a pKa near or below neutrality, such as, for example, MES, MOPSO, BES, MOPS, or ACES. Electro-blotting systems that include one or more of these buffers in the anodic gel matrix may optionally include a zwitterionic buffer with a pKa near or above neutrality in the cathode compartment, such as, for example, Tricine, Bicine, TAPS, TAPSO, or AMPSO.

An anion-forming buffer compound present in an anodic gel matrix of an electro-blotting system and absent from (or present in significantly reduced amounts in) the cathodic gel matrix of an electro-blotting system may be present at any concentration, but is typically present in the anodic gel matrix at a concentration of at least 10 mM, at a concentration of about 10 mM to about 1 Molar, at a concentration of about 20 mM to about 500 mM, and in some embodiments from about 50 mM to about 300 mM.

### Electrodes:

As described above, in some electro-blotting system embodiments, the anodic body of gel matrix is in contact with the anode. In some embodiments, the anode is attached to or juxtaposed with a second side of the anodic body of gel matrix, where the second side of the anodic body of gel matrix is opposite the first side of the anodic body of gel matrix that is in contact with the protein blotting membrane.

The anode can comprise any appropriate electrically conductive material, and can be of any shape, for example, the anode can be a layer that includes a non-metallic electrically conducting material, a coil structure, a mesh comprising a non-metallic electrically conducting material, a metal foil, a metal mesh and/or any combinations thereof. In certain embodiments, an electrically conducting electrode can comprise a nonconducting polymer coated with a conducting metal or nonmetal. An electrode of a nonconducting material coated with a conducting material can be in the form of a sheet, mesh, or other structure. An electrode can also comprise one or more electrically conducting non-metallic materials such as graphite, carbon, an electrically conducting polymer, and or any combinations thereof. The anode can comprise, for example, a conducting polymer, platinum, stainless steel, carbon, graphite, aluminum, copper, silver, or lead. In some embodiments, the anode comprises an electrochemically ionizable metal, such as, for example, copper, silver, or lead. The use of an electrochemically ionizable metal anode allows electrophoretic transfer to occur in the absence of oxygen evolution at the anode, as copper metal is preferentially ionized in place of water. In some embodiments, the anode may include a disposable copper electrode. In other embodiments, an anode may include aluminum. In some embodiments, an anode may be a disposable aluminum electrode.

It is also possible, in accordance with another embodiment of the invention, to deposit or coat silver metal (using various different metal deposition methods) on an electrically conducting substrate (such as, but not limited to a copper mesh or grid or a carbon or graphite based fabric, or even a thin layer of an electrically conducting polymer). The methods that may be used to apply a silver metal coating to such electrically conducting electrodes may include, i.e., chemical vapor deposition (CVD) methods, silver coating by dipping the electrode in molten silver, electroplating methods, methods of spray coating using silver particles dispersed in a suitable adhesion enhancing composition or formulation, chemical deposition methods performed in an aqueous or non-aqueous solutions (such as, for example, immersing the conductive electrode in an ammoniacal silver nitrate solution including glucose, as is well known in the art of silver coated mirror forming), direct vacuum deposition of silver from a hot silver metal filament onto a target electrode, and the like. Thus, any suitable silver coating or deposition or application methods known in the art may be used in obtaining the silver metal coated electrode of the present invention.

In one embodiment, the cathodic body of gel matrix is in contact with the cathode. The cathode may be attached to or juxtaposed with a second side of the cathodic body of gel matrix, where the second side of the cathodic body of gel matrix is opposite the first side of the cathodic body of gel matrix that is in contact with carrier matrix. The cathode may include any appropriate conductive material, and can be of any shape, for example, the cathode can be a layer that includes a non-metallic electrically conducting material, a mesh comprising a non-metallic electrically conducting material, a metal foil, a metal mesh and/or combinations thereof. In certain embodiments, an electrically conducting electrode can comprise a nonconducting polymer coated with a conducting metal or nonmetal. An electrode of a nonconducting material coated with a

Conducting material can be in the form of a sheet, mesh, or other structure. An electrode can also comprise one or more electrically conducting non-metallic materials such as graphite, carbon, an electrically conducting polymer, and or any combinations thereof. The cathode can comprise, for example, a conducting polymer, platinum, stainless steel, carbon, graphite, aluminum, copper, silver, or lead. In some embodiments, the cathode is a disposable copper electrode. In other embodiments, the cathode can comprise palladium, which absorbs hydrogen gas produced at the cathode during electrophoretic transfer. In some embodiments, the cathode is a disposable aluminum electrode.

In some embodiments, the anode and cathode may have the same or similar length and width dimensions as the anodic body of gel matrix and cathodic body of gel matrix, respectively. The surface of an anode or cathode that is juxtaposed with or embedded in a body of gel matrix need not be continuous; for example, an electrode can be a wire mesh or coil structure. In such embodiments, the surface of an electrode in contact with or embedded in a gel matrix may be considered to be defined by the outer dimensions of the surface of the electrode structure that is juxtaposed with the gel matrix. In some embodiments, the anode surface juxtaposed with an anodic body of gel matrix contacts at least 50%, at least 60%, at least 70%, at least 80%, at least 90%, or at least 95% of the side of the anodic gel matrix it is juxtaposed with. The anode surface juxtaposed with an anodic body of gel matrix can have an area that is essentially the same as the surface area of the side of the anodic gel matrix it is juxtaposed with. For example, for a generally rectangular, oval, or round electrode and body of gel matrix, the length and width dimensions of the anode are within 20% of the length and width dimensions of the body of anodic gel matrix, within 10% of the length and width dimensions of the body of anodic gel matrix, such as within 5% of the length and width dimensions of the body of anodic gel matrix, within 2% of the length and width dimensions of the body of anodic gel matrix. In such embodiments, the anodic body of gel matrix may advantageously conform closely to or be larger than the length and width dimensions of the carrier matrix and blotting membrane being electro-blotted.

In some embodiments, a cathode surface juxtaposed with a cathodic body of gel matrix contacts at least 50%, at least 60%, at least 70%, at least 80%, at least 90%, or at least 95% of the side of the cathodic gel matrix it is juxtaposed with. The cathode surface juxtaposed with an cathodic body of gel matrix can have an area that is essentially the same as the surface area of the side of the cathodic gel matrix it is juxtaposed with. For example, for a generally rectangular, oval, or round electrode and body of gel matrix, the length and width dimensions of the cathode are within 20% of the length and width dimensions of the body of cathodic gel matrix, within 10% of the length and width dimensions of the body of cathodic gel matrix, such as within 5% of the length and width dimensions of the body of cathodic gel matrix, within 2% of the length and width dimensions of the body of cathodic gel matrix. In such embodiments, the anodic body of gel matrix may advantageously conform closely to or be larger than the length and width dimensions of the carrier matrix and blotting membrane being electro-blotted.

Thus, in certain embodiments a system is provided having an anode in contact with an anodic body of gel matrix which is in contact with one side of a protein blotting membrane, and a cathode in contact with a cathodic body of gel matrix which is in contact with one side of a carrier matrix, the opposite side of which is in contact with the protein blotting membrane on the anodic body of gel matrix and the anode. In some embodiments, the cathode, cathodic body of gel matrix, the anode, and the anodic body of the gel matrix have the same or nearly the same length and width dimensions.

In some embodiments, an anode, a cathode, or both may be provided as an integral part (meaning it is not detached by the user after each blotting procedure), of a power supply or apparatus that holds an blotting stack. In other embodiments, the anode or cathode may be separate from a power supply or apparatus. For example, an electrode may be a disposable electrode provided as part of an electrode assembly or separate from the body of gel matrix. An electrode provided separately from a gel matrix may be attached to an electro-blotting apparatus after which a body of gel matrix may be fitted to the apparatus such that it contacts the electrode, or both electrode and body of gel matrix can be positioned in a holder, such as a tray or cage, that can be attached to or fitted to an electro-blotting apparatus.

In some embodiments, the anode, cathode, or both is provided as part of an electrode assembly attached to a body of gel matrix, for example, the anode or cathode can be attached using fasteners or holders that position the electrode against a body of gel matrix. In certain embodiments, an anode or cathode is at least partially embedded in the anodic body of gel matrix. For example, a body of gel matrix can be made by pouring unsolidified gel components over an electrode or by using gel extrusion techniques, such that the electrode becomes partially coated or embedded on at least one side by gel matrix. In certain embodiments, the body of gel matrix is positioned against the conducting electrode in a plastic tray before and during electrophoretic transfer. The plastic tray has at least one region that comprises conductive material for providing electrical connection between the electrode and an electrical contact of a power supply or source.

Returning to FIG. 1A, an electro-blotting system may include power supply 109 having first electrical contact 110 for contacting anode 105, and second electrical contact 111 for contacting cathode 107. The power supply can have a base for positioning an blotting stack during a blotting procedure. In some embodiments, an anode, a cathode, or both may be integral to a power supply of the system. In other embodiments, an anode, a cathode or both may be separate from but coupleable to the power supply though electrical connections. Power supply 110 and electrical connections 110 and 111 may be configured so as to allow the application of an electrical current between the top stack and the bottom stack.

In some embodiments, blotting stack 100 may include carrier matrix 112 positioned between the anodic and cathodic assemblies as depicted in FIG. 1A. In an embodiment, the surface of carrier matrix 112 proximal to the anodic stack may be juxtaposable with the surface of anodic gel matrix 106 that is opposite to the surface coupled to anode 105, whereby such juxtaposition occurs via a blotting membrane interspersed therebetween as discussed in detail below. Likewise, the surface of carrier matrix 112 proximal to the cathodic stack may be juxtaposable with the surface of cathodic gel matrix 108 that is opposite to the surface coupled to cathode 107. Such configuration ensures the flow of electrical current through the carrier matrix during use. The dimensions of a carrier matrix as presently contemplated may be substantially similar to the dimensions of the upper stack and/or the lower stack. Alternatively, the dimensions of the carrier matrix may be smaller than the dimensions of the upper stack and/or the lower stack.

In an embodiment, the length and width of the carrier matrix may be selected such that a surface of a protein blotting membrane juxtaposed thereto is in contact with the surface of the carrier matrix. Typically, the dimensions of the carrier matrix will be substantially similar to the dimensions of the protein blotting membrane and/or to the gel matrix bodies. In an embodiment, the length of at least one side of a carrier matrix will be in the range of about 2 cm to about 25 cm, about 5 cm to about 20 cm, about 8 cm to about 15 cm, or about 10 cm to about 12 cm. Likewise, the length of another side of a carrier matrix will be in the range of about 2 cm to about 25 cm, about 5 cm to about 20 cm, about 8 cm to about 15 cm, or about 10 cm to about 12 cm.

A carrier matrix, as used herein, will be in the form of a sheet having a thickness of less than about 5 mm, less than about 3 mm, less than about 2 mm, less than about 1 mm or less than about 0.5 mm. In some non-limiting embodiments, at least one surface or both surfaces of a carrier matrix sheet may be substantially smooth. During use, the smooth surface of the carrier matrix will be juxtaposed with the surface of a protein blotting membrane having the biomolecular sample coupled thereto. Doing so may substantially enhance even transfer of proteins (e.g., antibodies, blocking reagents etc.) from the carrier matrix to the protein blotting membrane and reduce the likelihood of obtaining pixelated of “grainy” bands in experimental results.

In an embodiment, a carrier matrix sheet may be made of fibers or microfibers of a naturally occurring material, a synthetic material or a composite thereof. A carrier matrix suitable for use in an electro-blotting system will be made of a material that is able to absorb between about 0.2 ml to about 5 ml, between about 0.5 ml to about 2.5 ml, or between about 0.75 ml to about 1.5 ml of an aqueous proteinaceous or hybridization solution or buffer. In certain non-limiting embodiments, a carrier matrix may be made of an absorbent material that is capable of substantially reversibly absorbing an aqueous proteinaceous or hybridization composition and has minimal intrinsic protein binding potential. Any material having such properties may be employed in the practice of the present invention without limitation. Ideally, a suitable carrier matrix may be made of a material that, when immersed in a large volume of an aqueous solution, releases into the aqueous solution at least about 20%, at least about 35%, at least about 45%, at least about 50%, at least about 55%, at least about 60%, at least about 65%, at least about 75%, at least about 80%, at least about 85%, at least about 90% or at least about 95% of a proteinaceous or hybridization composition absorbed thereon within 1 minute, within 2 minutes, within 3 minutes, within 5 minutes or within 10 minutes of being immersed in the aqueous solution. Such a determination is well within the skill level of a practitioner having ordinary skill in the art and may be readily made by such a practitioner without undue experimentation. For example, to determine whether a test material may be suitable for use as a carrier matrix in accordance with the present embodiments, the skilled artisan may absorb a known amount of a proteinaceous or hybridization solution (containing a readily measurable protein such as, e.g., BSA, IgG, casein, actin, GAPDH etc.) on the test material and immerse the test material in a known volume of an aqueous buffer (e.g., PBS). At various time intervals, samples of the aqueous buffer may be collected and the concentration of the protein released from the test material may be determined (for example using ELISA, quantitative western blot, or any other similar technique). Alternatively, a suitable carrier matrix may be made of a material that releases at least about 10%, at least about 20%, at least about 35%, at least about 45%, at least about 50%, at least about 55%, at least about 60%, at least about 65%, at least about 75%, at least about 80%, at least about 85%, at least about 90% or at least about 95% of a proteinaceous or hybridization composition absorbed thereon within 1 minute, within 2 minutes, within 3 minutes, within 5 minutes or within 10 minutes of passing an electric current of at least 20 V, at least about 15 V, at least about 10V, at least about 5 V or at least about 3 V across the membrane.

By way of non-limiting example, exemplary materials suitable for use in the manufacture of carrier matrix sheets according to the present invention may include polyester fibers, polycarbonate fibers, glass microfibers, hydrophilic cellulose fibers, cellulose acetate fibers, hydroxylated polyamide fibers (e.g., LOPRODYNE®), polyethersulfone fibers, acrylic co-polymer fibers, mixed cellulose ester fibers, modified poly(tetrafluoroethene) (PTFE), filter paper, felt, or combinations or composites thereof.

In some embodiments, a carrier matrix may include one or more sheets of blotting paper. In an embodiment, a carrier matrix may include one or more sheets on filter paper (such as WHATMAN® filter paper). In an embodiment, a carrier matrix may include one or more sheets of synthetic microfibers. Synthetic microfibers used in such sheets may include polyester microfibers or polyamide microfibers or a blend of polyester and polyamide microfibers. An exemplary polyester/polyamide microfiber sheet suitable for the present purpose may be obtained commercially from Sadovsky Houshold products, Ltd. Ashdod, Israel.

Returning now to FIG. 1A, in an embodiment blotting stack 100 may further include an optional second carrier matrix 113. Second carrier matrix 113 may be substantially similar in size, shape and composition to carrier matrix 112. In some embodiments, second carrier matrix 113 may be assembled in blotting stack 100 at the same time as carrier matrix 112. For example, carrier matrix 112 may be proximal to the bottom stack, and second carrier matrix 113 may be proximal to the top stack as depicted.

In alternate embodiments, carrier matrix 112 and second carrier matrix 113 may be included in stack 100 sequentially. For example, carrier matrix 112 may be used in stack 100 first. After current has been applied and electrophoretic transfer of proteins (e.g., blocking reagents and at least a primary antibody) absorbed thereon is achieved, carrier matrix 112 may be discarded and replaced by second carrier matrix 113 having different proteins (e.g., optional blocking reagent and a secondary antibody) absorbed thereon.

Turning now to FIG. 1B, an alternate embodiment of an electro-blotting system is shown. In this embodiment, an electro-blotting system may include tray 115. Tray 115 may be sized to accept one or more components of stack 100, such as bottom stack 102. Tray 115 may be a disposable plastic tray.

An electro-blotting system may include protein blotting membrane 116 supplied by the user of the electro-blotting system. In an embodiment, membrane 116 may have substantially the same dimensions as carrier matrices 112 and 113. In an embodiment, the dimensions of membrane 116 may be smaller than the dimensions of carrier matrices 112 and 113. In an embodiment, membrane 116 may have substantially the same dimensions as gel matrix bodies 106 and 108. In an embodiment, the dimensions of membrane 116 may be smaller than the dimensions of gel matrix bodies 106 and 108. Membrane 116 may be made of any material capable of having constituent molecules of a biomolecular sample substantially immobilized thereon. By way of non-limiting example, membrane 116 may include paper, a cellulose-based blotting membrane (such as but not limited to cellulose nitrate or cellulose acetate), a nitrocellulose (NC)-based membrane, a polyamide-based membrane, or polyvinylidene difluoride (PVDF)-based membrane, or activated or derivatized versions of these (such as, for example, surface-charged derivatives), or combinations or composites thereof.

Membrane 116 as depicted in FIG. 1B includes first side 117 and second side 118. In some embodiments, membrane 116 may include biomolecular sample 119 coupled to one side thereof (e.g., side 117) prior to its use in an electro-blotting system according to the present embodiments. Sample 119 may include proteins, nucleic acids (e.g., DNA or RNA), carbohydrates, lipids or any combinations or composites thereof. Sample 119 may be derived, for example from a cell or tissue lysate or other biological sample such as serum, may be a complex mixture of biomolecules, or may be purified or at least partially purified.

In some embodiments, components of sample 119 are resolved by electrophoresis (e.g., SDS-PAGE, agarose gel electrophoresis, etc.) prior to coupling the sample to membrane 116. Sample 119 may be coupled to membrane 116 using any art-recognized technique for doing so, without limitation. Such techniques may also be referred to in the art as “electrophoretic transfer” or more simply “transfer”. A variety of transfer techniques, including wet, dry or semi-dry transfer techniques, are know to those skilled in the art. Exemplary though non-limiting transfer methods suitable for use in accordance with the present invention are described, e.g., in the review article entitled “Protein Blotting: A review” by B. T. Kurien and R. H. Scofield published in J. of Immunological methods, Vol. 274, pp. 1-15 (2003), which describes, i.a., various protein blotting methods including wet and semi-dry electro-blotting methods, in U.S. Pat. Nos. 5,482,613, 5,445,723, 5,356,772, 4,889,606, 4,840,714, 5,013,420, and US Published Application 2002157953 which disclose, i.a., various types of apparatuses and methods for performing wet and semi-dry electrophoretic transfer, and in U.S. Published Applications 20060278531 and 20060272946 which describe dry electro-blotting systems for dry blotting gels and methods for using same, in which the system includes an electro-blotting transfer stack. The above-cited references are hereby expressly incorporated by reference in their entirety as though fully set forth herein. Other methods well known to one skilled in the art for coupling a biomolecular sample to a solid membrane support include, though are not limited to, dot blotting, spotting, vacuum transfer and capillary transfer.

Returning to FIG. 1B, membrane 116 may be positioned between lower stack 102 and carrier matrices 112/113 such that side 118 thereof is substantially juxtaposed with gel matrix body 106 and such that side 117 and sample 119 are substantially juxtaposed with carrier matrix 112 as shown.

Turning now to FIG. 1C, an electro-blotting system according to yet another embodiment is shown. This embodiment incorporates the enhancements of the embodiments depicted in FIGS. 1A and 1B, except that tray 115 has been removed. In this embodiment, blotting stack 100, which includes anode 105 and anodic gel matrix 106 of bottom stack 102, cathode 107 and cathodic gel matrix 108 of top stack 104, carrier matrices 112 and 113 and membrane 116 is assembled and the elements thereof are appropriately juxtaposed as described above in detail and incorporated herein.

In an embodiment, an electro-blotting system may include housing 120 having top portion 121 and bottom portion 122. In some embodiments, top portion 121 may be coupled to power source 109 through electrical coupling 111, and bottom portion 122 may be coupled to power source 109 through electrical coupling 110, thereby allowing a current to be passed between the top and bottom portions of the housing. Any suitable housing may be employed in the practice of such embodiments. An exemplary housing that is particularly well suited to the practice of the present invention is the IBLOT™ system (Invitrogen Corporation, Carlsbad, Calif.), described in U.S. Published Applications 20060278531 and 20060272946. In an embodiment, the dimensions of housing 120 may be sized such that blotting stack 100, when assembled, is positionable between top portion 121 and bottom portion 122. In an embodiment, cathode 107, anode 109, or both cathode 107 and anode 109 are in electrical communication with top portion 121 and bottom portion 122, respectively, thereby allowing a user to pass an electric current between the anode and the cathode.

In an embodiment, an electro-blotting system may optionally include sponge 123. Sponge 123 may be disposable or may be multi-use. In an alternate embodiment, sponge 123 may be replaced by one or more filter papers. Without being bound by any particular theory or mechanism, sponge 123 may be included in the system to absorb extraneous liquid produced when housing 120 is assembled, and pressure 130 is applied to the blotting stack during use. Additionally, sponge 123 may also served to increase pressure 130 in the indicated direction, which helps to ensure that all juxtaposed surfaces remain in constant and/or even contact during use. In an embodiment, sponge 123 may include clip 124. In one non-limiting embodiment, clip 124 may be juxtaposed with at least two opposite surfaces thereof and connected through central portion 125. In another non-limiting embodiment, clip 124 may pass entirely through the body of sponge 123. Clip 124 may be made entirely or partially of any electrically conductive material, such as gold, copper, silver, aluminum, alloys thereof, stainless steel or an electrically conductive polymer or polymer coating, so as to ensure electrical continuity between the top portion of housing, the cathode, the blotting stack, the anode, and the bottom portion of the housing during use.

### Methods for Performing Electrically-Enhanced Analyte Detection:

Having now described the components of an electro-blotting system and how they are assembled relative to one another during use, methods for using the system to perform an blotting procedure according to various embodiments will now be described.

It should be noted that, although the following description of methods for using the presently embodiment electro-blotting system makes reference to various steps involved in performing the procedure, it will be readily apparent to one skilled in the art that one or more alternative methods or procedures are equally possible, and are included within the scope of the present disclosure. It should also be noted that failure to specifically recite any one or more alternative methods does not exclude inclusion of such methods within the scope of the present invention, as long as such methods fall within the general spirit and scope of the presently described methods, and make use of one or more of the presently described systems, methods or apparatuses, such as will be readily apparent to a person having ordinary skill in the art to which the present application pertains. For example, although steps of the forgoing description are presented in a defined order, the skilled artisan will readily recognized that certain of those steps may, depending on the specific context thereof, be performed outside of the sequence explicitly set forth below. By way of non-limiting example, in some instances stock solutions, diluted antibodies, wash buffers, and the like may be prepared in advance of the procedure and set aside for later use. Additionally, it will be appreciated that one or more additional washing steps, de-bubbling steps, blocking steps, etc. may be performed and are included within the scope of the invention, even though not explicitly described in detail below.

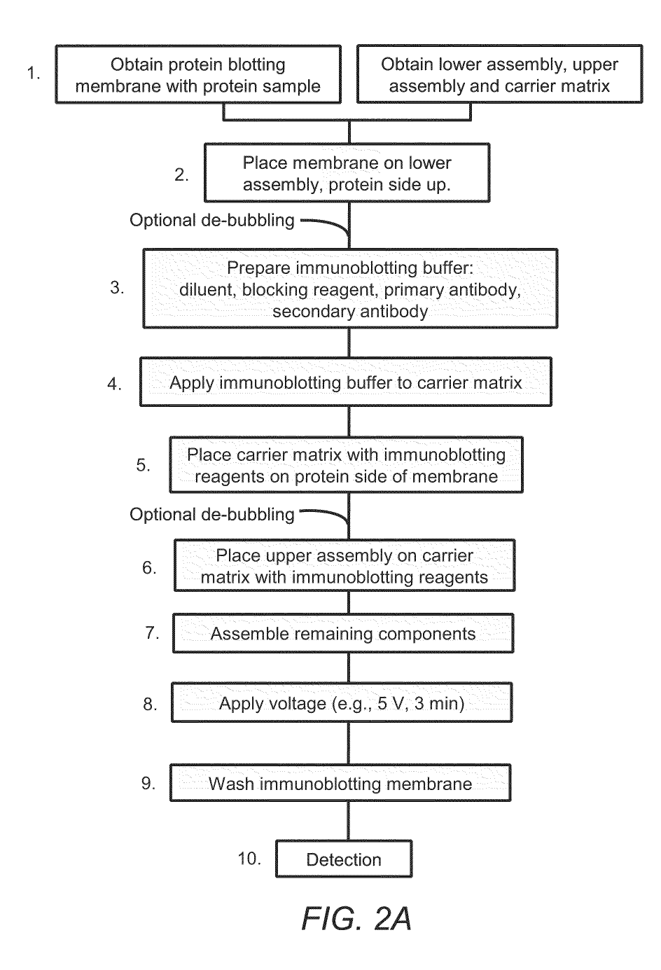

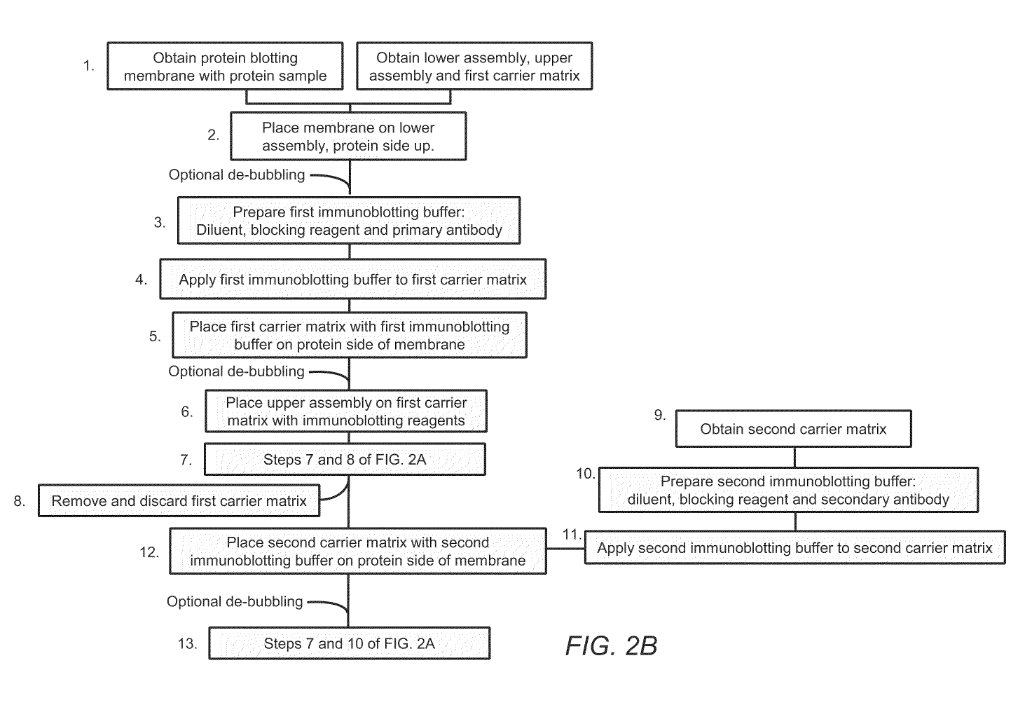

Turning to FIG. 2, a method for performing an electro-blotting procedure in accordance with one embodiment is outlined. To perform an electro-blotting procedure, a user may obtain a protein blotting membrane having a biomolecular sample coupled to a surface thereof. Typically, a sample is obtained and resolved by electrophoresis (e.g., SDS-PAGE) after which the resolved molecules are transferred or immobilized to an appropriate solid support. An appropriate membrane is described above. A user may also obtain a lower assembly having an anode and an anodic gel matrix body as described in detail above. The protein blotting membrane may be placed on the lower assembly such that the surface of the membrane lacking the biomolecular sample is juxtaposed with the surface of the anodic gel matrix body opposite the anode as shown in FIG. 1C. An optional de-bubbling step may be performed to remove any air pockets between the protein blotting membrane and the anodic gel matrix.

In an embodiment, a user may prepare a blotting buffer. A blotting buffer will typically include a diluent. A diluent may be prepared by the user prior to use, may be obtained commercially, or may be supplied as part of a kit along with various components of the presently described system. A diluent may include a physiologically acceptable aqueous solution having a pH in the range of about 4 to about 9, or from about 5 to about 8, or from about 6 to about 7.5, and typically having at least one buffering agent such as, e.g., phosphate buffer, bicarbonate, TAPS, Bicine, Tris, Bis-Tris, Tricine, HEPES, TES, MOPS, PIPES, Cacodylate, MES, acetate, ADA, ACES, cholamine, BES, acetamidoglycine or glycinaide present therein. Exemplary buffers suitable for use as diluents may include, though are not limited to, e.g., PBS, Hank's solution, TBS, TE, TEN, or the like. Optionally, a diluent may include a detergent. Suitable detergents may include non-ionic, non-denaturing detergents such as, e.g., Triton X-100, Triton X-114, NP-40, Brij-35, Brij 58, Tween-20, Tween-80, octyl glucoside and octylthio glucoside. A diluent may contain from about 0.01 vol % to about 5 vol. %, from about 0.05 vol % to about 2 vol. %, from about 0.1 vol % to about 1.5 vol. %, or from about 0.5 vol % to about 1 vol. % of a suitable detergent. During a typical procedure, a user may prepare enough blotting buffer to absorb onto a carrier matrix. A sufficient amount of a blotting buffer will be sufficient to soak the carrier matrix. Typically, the user will prepare at least 1 ml, at least 2 ml, at least 5 ml, at least 10 ml, or at least 20 ml of an appropriate blotting buffer. This volume may be used during one or more steps of the procedure.

Returning to FIG. 2A, a blotting buffer may include one or more blocking reagents. Blocking reagents may be used to block non-specific sites on a protein blotting membrane prior to probing thereof with one or more primary or one or more secondary antibodies. Blocking reagents may be dispersed or dissolved a diluent as described above. Blocking reagents may be prepared by a user and added to a blotting buffer prior to use of the electro-blotting system. Alternatively, stock preparations of blocking reagents may be prepared by the user in advance and added to a diluent or a blotting buffer immediately prior to use thereof. A stock preparation of a blocking reagent may be, for example, up to 20×, up to 10×, up to 5×, up to 2× or up to 1.5× the concentration typically used in protein blotting procedures. A stock solution may be prepared in any appropriate buffer system. Alternatively, blocking reagents may be supplied as a component of the diluent and sold commercially as part of an electro-blotting kit.

Any suitable blocking reagent may be employed for use with the presently described electro-blotting system without limitation. A variety of suitable blocking reagents are known in the art and may include, though are not limited to, whole serum, fractionated serum, bovine serum albumin, casein, soy protein, non-fat milk, gelatin, fish serum, goat immunoglobulin, rabbit immunoglobulin, mouse immunoglobulin, rat immunoglobulin, horse immunoglobulin, human immunoglobulin, pig immunoglobulin, chicken immunoglobulin, whey proteins, rice proteins, algae proteins or synthetic blocking reagents, such as those that may be obtained commercially form, e.g., BioFX Laboratories, Kem-En-Tec Diagnostics or GeneWay Biotech. A variety of commercially available pre-prepared blocking reagents are available in the art, all of which may be employed in accordance with the present systems and methods. Such commercially available blocking reagents include, though are not limited to, e.g., WesternBreeze, I-BLOCK, Blocklt, PerfectBlock, Synthetic Blocking Buffer (BioFX Labs), Gelantis BetterBlock, SeaBlock, Starting Block and Protein-Free Blocking Buffer (Pierce). In an embodiment, the amount of a blocking reagent present in a blotting buffer may be in the range of about 0.1 wt. % to about 50 wt. %, about 1 wt. % to about 40 wt. %, about 2.5 wt. % to about 25 wt. %, about 5 wt. % to about 15 wt. % or about 10 wt %. In an embodiment, the amount of a blocking reagent present in a blotting buffer may be up to about 75 mg/ml, up to about 50 mg/ml, up to about 40 mg/ml, up to about 30 mg/ml, up to about 20 mg/ml, up to about 15 mg/ml, up to about 10 mg/ml up to about 5 mg/ml, up to about 2.5 mg/ml, up to about 1 mg/ml, up to about 0.5 mg/ml, up to about 0.25 mg/ml or up to about 0.1 mg/ml.

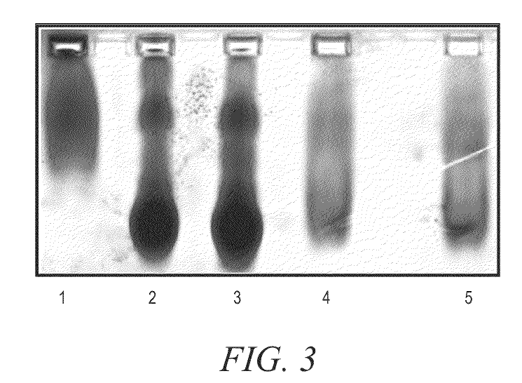

In an embodiment, a suitable blocking reagent may have an inherent negative charge. Having an inherent negative charge may ensure that the blocking reagent migrates from the carrier matrix to the surface of the protein blotting membrane during use. A variety of blocking reagents are known to have an inherent negative charge and the determination of such is well within the skill level of a practitioner having ordinary skill in the art. One exemplary though non-limiting method that the skilled practitioner may use to determining whether a particular blocking reagent has an inherent negative charge suitable enough such that the blocking reagent may be electrophoresed onto a membrane using the instant systems, methods and kits is illustrated in FIG. 3 and discussed in detail below. Alternatively, a protein blocking reagent may be engineered to impart a negative charge thereto. Such may be accomplished, by way of example, by incorporating a plurality of negatively charged amino acids in the polypeptide sequence of a protein. A wide variety of recombinant DNA techniques may be used to incorporate negatively charged amino acids in a particular protein, and such techniques are within the skill level of a practitioner having ordinary skill in the art.

In an embodiment, a blotting buffer may include a primary antibody in an appropriate diluent. In an embodiment, the blotting buffer may include a blocking reagent as described above and incorporated herein, in combination with a primary antibody. The concentration of primary antibody in the blotting buffer will of course vary, depending on the specific primary antibody being used, the context in which the antibody is being used, and various other properties inherent in the antibody. Typically, the concentration of the primary antibody will be 1:10 to 1:20,000, 1:100 to 1:15,000, 1:1,000 to 1:10,000 or 1:1,500 to 1:5,000.

The primary antibody may be a user-defined antibody. The antibody may be directed against a user defined antigen. The antibody may be purchased commercially or may be made by the user. The antibody may be a polyclonal antibody or a monoclonal antibody. A monoclonal antibody may be raised in mouse or in rat. A monoclonal antibody may be IgG (IgG1, IgG2a, IgG2b, IgG3), IgM, IgA, IgD and IgE subclasses. A polyclonal antibody may be raised in rabbit, mouse, rat, hamster, sheep, goat, horse, donkey or chicken. In an embodiment, an antibody may be derived from human serum. A human antibody may be at least partially or fully purified. Methods of preparing and purifying antibodies are widely known in the art. General guidance in the production and use of various antibody preparations may be found, for example in the reference texts Harlow et al., 1989, Antibodies: A Laboratory Manual, Cold Spring Harbor, N.Y., Harlow et al., 1999, Using Antibodies: A Laboratory Manual, Cold Spring Harbor Laboratory Press, NY, and Harlow, et al., 1988, In: Antibodies, A Laboratory Manual, Cold Spring Harbor, N.Y., all of which are hereby expressly incorporated by reference.

In some embodiments, a primary antibody may be a “loading control antibody”. The loading control antibody may be provided by the user or may be provided commercially as part of the presently descried system (i.e., a kit, such as is described in detail below). Exemplary though non-limiting loading control antibodies that may be used or supplied with the presently described systems and methods may include antibodies directed against actin, tubulin, histone, vimentin, lamin, GAPDH, VDAC1, COXIV, hsp-70, hsp-90 or TBP.

In an embodiment, a blotting buffer may include a secondary antibody in an appropriate diluent. A secondary antibody may be coupled to a detection means. Of course, it will be readily apparent to the skilled artisan that what constitutes a suitable secondary antibody depends on the identity of the one or more primary antibodies used in the steps described above. A secondary antibody will be selected to bind to at least a portion of the primary antibody. Selection of an appropriate antibody further depends on the methods that will be used to detect the signal in later steps. If an investigator is using chemiluminescent techniques to detect an analyte, then a suitable secondary antibody may be coupled to a peroxidase enzyme or an alkaline phosphatase. If an investigator is using calorimetric techniques to detect an analyte, then a suitable secondary antibody may be coupled to an alkaline phosphates or a peroxidase enzyme. If an investigator is using fluorometic techniques to detect an analyte, then a suitable secondary antibody may be coupled to a fluorophore. Optionally, a secondary antibody may be coupled to one or more biotin moieties and the detection molecule (e.g., peroxidase, phosphates, fluorophore etc.) may be coupled to avidin. Using such a biotin/avidin system may, in some cases amplify weak detection signals. Typically, the concentration of the secondary antibody will be 1:10 to 1:20,000, 1:100 to 1:15,000, 1:1,000 to 1:10,000 or 1:1,500 to 1:5,000.

A suitable secondary antibody for use with the presently described systems and methods may be raised, for example, in rabbit, mouse, rat, hamster, pig, sheep, goat, horse, donkey, turkey or chicken. The secondary antibody will typically be raised in a different species than the species in which the primary antibody was raised. The secondary antibody will be generated such that it recognizes and binds to a portion of the primary antibody. The secondary antibody may be at least partially affinity purified. The secondary antibody may be directed against mouse IgG, mouse IgA, mouse IgM, rat IgG, rat IgA, rat IgM, rabbit IgG, rabbit IgA, rabbit IgM, hamster IgG, hamster IgA, hamster IgM, goat IgG, goat IgA, goat IgM, horse IgG, horse IgA, horse IgM, sheep IgG, sheep IgA, sheep IgM, donkey IgG, donkey IgA, donkey IgM, chicken IgG, chicken IgA, chicken IgM, chicken IgY, human IgG, human IgA, or human IgM. A secondary antibody may be coupled to one or more detection molecules such as, by way of example, alkaline phosphatase, peroxidase, biotin, a fluorophore or Qdot nanocrystals, as discussed above.

In an embodiment, the blotting buffer may include a blocking reagent as described above and incorporated herein, in combination with a secondary antibody.

In an embodiment, a blotting buffer may include an appropriate blocking reagent as described above and incorporated herein, in combination with a primary antibody and a secondary antibody. The concentrations of the primary antibody and the secondary antibody may be 1:10 to 1:20,000, 1:100 to 1:15,000, 1:1,000 to 1:10,000 or 1:1,500 to 1:5,000, though different concentrations may be used depending on the identity and properties of the antibodies selected by the end user for use with the presently described systems and methods.

In an alternate embodiment, in situation where the secondary antibody used was a biotinylated antibody, a blotting buffer may include peroxidase- or phosphatase-coupled avidin/streptavidin. Typically, the concentration of peroxidase- or phosphatase-coupled avidin/streptavidin present in an blotting buffer may be 1:10 to 1:20,000, 1:100 to 1:15,000, 1:1,000 to 1:10,000 or 1:1,500 to 1:5,000.

Returning now to FIG. 2A, the blotting buffer containing the blocking reagent, the primary antibody, the secondary antibody, or the primary and the secondary antibodies may be absorbed onto the carrier matrix described above. An appropriate volume of the blotting buffer to absorb onto the carrier matrix will be a sufficient volume so that the entire body of the carrier matrix is substantially soaked with the blotting buffer, but not overly saturated so as to cause excess buffer to leak from the carrier matrix. What constitutes a sufficient volume of blotting buffer to achieve this end will of course vary depending on the dimensions, thickness, and capacity of the matrix in question. Typically, such a volume will be up to about 1 ml, up to about 1.5 ml, up to about 2 ml, up to about 2.5 ml, up to about 4 ml, up to about 6 ml, up to about 8 ml, or up to about 10 ml of an blotting buffer. To accomplish this, the carrier matrix may be placed in an appropriate vessel (e.g., an appropriately shaped and sized Petri dish to accommodate the carrier matrix) and the blotting buffer with the blocking agent, the primary antibody, the secondary antibody, or various combinations thereof may be directly absorbed onto the matrix. The soaked carrier matrix is then placed on top of the blotting membrane, such that the surface of the blotting membrane having the biomolecular sample coupled thereto is substantially contacted with the soaked carrier matrix. An optional de-bubbling step as described above may be performed.

In an embodiment, the carrier matrix placed on the blotting membrane may have the blocking reagent, the primary antibody and the secondary antibody absorbed thereon.

In an alternate embodiment, the carrier matrix may have the blocking reagent absorbed thereon, and a second carrier matrix having the primary antibody absorbed thereon may be prepared and placed over the first carrier matrix.

In another alternate embodiment, the carrier matrix may have the blocking reagent and the primary antibody absorbed thereon, and a second carrier matrix having the secondary antibody absorbed thereon may be prepared and placed over the first carrier matrix.

In yet another alternate embodiment, the carrier matrix may have the blocking reagent absorbed thereon, a second carrier matrix having the primary antibody absorbed thereon may be prepared and placed over the first carrier matrix, and a third carrier matrix having the secondary antibody absorbed thereon may be prepared and placed over the second carrier matrix.

While the above description makes specific reference to “a primary antibody” and “a secondary antibody”, it will be readily appreciated by one skilled in the art that the use of more than one primary antibody and the use of more than one secondary antibody are equally contemplated and may be used in the practice of the presently described embodiments. By way of example, a blotting buffer may be prepared and may contain a plurality of primary antibodies. One or more of such primary antibodies may be loading control antibodies, are all may be directed against user-determined antigens. Such embodiments are exemplified in greater detail below.

Returning to FIG. 2A, the user may obtain an upper assembly having a cathodic gel matrix body and an electrode coupled thereto. The upper assembly may be placed over the one or more carrier matrices such that the surface of the cathodic gel matrix is substantially contacted with the surface of the carrier matrix. The upper assembly, or a portion thereof, may be integral to the housing or may be separate from the housing. The user assembled the remaining components of the system as depicted in FIG. 1C and applies an electric current such that the current passes between the cathode and the anode. The current may be applied for up to about 20 minutes, up to about 15 minutes, up to about 10 minutes, up to about 5 minutes, or up to about 3 minutes. The applied current may be up to about 25V, up to about 20V, up to about 15V, up to about 10V, up to about 5V minutes, or up to about 3V minutes. Applying an electric current to the system may cause at least a portion of the proteinaceous or hybridization composition (e.g., the blocking reagent, the primary antibody and the secondary antibody) to migrate from the one or more carrier matrices to the protein blotting membrane, where the appropriate antigen-antibody binding reactions may occur.

When the current is terminated, the system may be disassembled and the protein blotting membrane is retrieved and subjected to at least one washing step. The washing steps are typically performed to remove any unbound secondary antibody and thereby increase the signal-to-noise ratio of downstream collected data. The membrane may be immersed in at least 2 ml, at least 5 ml, at least 10 ml, or at least 20 ml of an appropriate buffer (e.g., one of the buffer systems described above and incorporated herein) optionally in the presence of a detergent. Each washing step is typically performed for at least 1 min, at least 2 min, at least 5 min or at least 10 min, though longer or shorter washes are permissible. During a typical procedure, three 5 minute washes are performed.

Following the washing steps, the protein blotting membrane is subjected to a detection step. What constitutes a suitable detection means will of course depend on the identity and properties of the secondary antibody being used, as will be evident to the skilled artisan. By way of example, if the secondary antibody is coupled to a peroxidase enzyme, enhanced chemiluminescence (ECL) may be used to detect the presence of a test antigen. ECL is an art-recognized technique for a variety of detection assays in biology. A horseradish peroxidase enzyme (HRP) is tethered to the molecule of interest (usually through labeling an immunoglobulin that specifically recognizes the molecule). This enzyme complex then catalyzes the conversion of the ECL substrate into a sensitized reagent in the vicinity of the molecule of interest, which on further oxidation by hydrogen peroxide, produces a triplet (excited) carbonyl which emits light when it decays to the singlet carbonyl. ECL allows detection of minute quantities of an antigen. Proteins can be detected down to femtomole quantities, well below the detection limit for most assay systems.

Turning now to FIG. 2B, a method of performing an electro-blotting procedure according to an alternate embodiment is outlines. This embodiment differs from the embodiment shown in FIG. 2A and discussed above, in that the previous embodiment is performed as a single step, i.e., both the primary and the secondary antibodies are applied to one or more carrier matrices, which are then assembled into the system as described above. An electric current is applied to the system such that the primary and the secondary antibodies migrate from the carrier matrices to the protein blotting membrane, where at least the primary antibody binds to its target antigen if such an antigen is present on the surface of the blotting membrane, and the secondary antibody binds to the primary antibody. According to the alternate embodiment discussed below, the electro-blotting procedure is performed in at least two steps. For example, the primary antibody is applied to a carrier matrix, which is then assembled in to the system as described above. A voltage is applied and the primary antibody binds to its target on the surface of the protein blotting membrane. Following this, the carrier matrix is removed, and the secondary antibody is applied to a second carrier matrix, which is assembled into the system, and a voltage is applied such that the secondary antibody migrates to the surface of the protein blotting membrane, where it binds to the corresponding primary antibody. It should be noted that in this embodiment, all reagents, solutions, components, volumes, and concentrations are identical to those specified above with regard to the single-step process, the difference being the order with which the components are used and the number of steps employed to accomplish the procedure.

To perform a 2-step electro-blotting procedure, a user may obtain a protein blotting membrane having a biomolecular sample coupled to a surface thereof. Typically, a sample is obtained and resolved by electrophoresis (e.g., SDS-PAGE) after which the resolved molecules are transferred or immobilized to an appropriate solid support. An appropriate membrane is described above. A user may also obtain a lower assembly having an anode and an anodic gel matrix body as described in detail above. The protein blotting membrane may be placed on the lower assembly such that the surface of the membrane lacking the biomolecular sample is juxtaposed with the surface of the anodic gel matrix body opposite the anode as shown in FIG. 1C. An optional de-bubbling step may be performed to remove any air pockets between the protein blotting membrane and the anodic gel matrix.

In an embodiment, a user may prepare a first blotting buffer. The first blotting buffer may include an appropriate diluent, a blocking reagent and a primary antibody.

The first blotting buffer may be absorbed onto a first carrier matrix as described above, after which the carrier matrix is placed over the protein blotting membrane such that the surface of the membrane having the biomolecular sample coupled thereto is juxtaposed with the soaked first carrier matrix. An optional debubbling step may be performed to remove any pockets of air between the soaked carrier matrix and the protein blotting membrane.

Returning to FIG. 2B, the user may obtain an upper assembly having a cathodic gel matrix body and an electrode coupled thereto. The upper assembly may be placed over the first carrier matrix such that the surface of the cathodic gel matrix is substantially contacted with the surface of the carrier matrix. The user assembles the remaining components of the system as depicted in FIG. 1C and applies an electric current such that the current passes between the cathode and the anode. The voltage may be applied for up to about 20 minutes, up to about 15 minutes, up to about 10 minutes, up to about 5 minutes, or up to about 3 minutes. The applied voltage may be up to about 25V, up to about 20V, up to about 15V, up to about 10V, up to about 5V, or up to about 3V. Applying an electric current to the system may cause at least a portion of the proteinaceous or hybridization composition (e.g., the blocking reagent and the primary antibody or nucleic acid probe) to migrate from the first carrier matrix to the protein blotting membrane, where the appropriate antigen-antibody binding reactions may occur.

When the voltage is terminated, the system may be at least partially disassembled so that the at least partially spent first carrier matrix may be retrieved and optionally discarded. The remaining components are retained for an additional round of electro-blotting. At this point, the user may obtain a second carrier matrix. The properties, composition and dimensions of the second carrier matrix may be identical to those described above and incorporated herein.

In an embodiment, the user may prepare a second blotting buffer. The second blotting buffer may include an appropriate diluent, a secondary antibody and optionally a blocking reagent. The second blotting buffer may be absorbed onto the second carrier matrix as described above, after which the second carrier matrix is placed over the protein blotting membrane such that the surface of the membrane having the biomolecular sample coupled thereto is juxtaposed with the soaked second carrier matrix. An optional debubbling step may be performed to remove any pockets of air between the soaked second carrier matrix and the protein blotting membrane.

In an embodiment, the retained upper assembly having a cathodic gel matrix body and an electrode coupled thereto may be placed over the second carrier matrix such that the surface of the cathodic gel matrix is substantially contacted with the surface of the second carrier matrix. The user assembles the remaining components of the system as depicted in FIG. 1C and applies an electric voltage such that the current passes between the cathode and the anode. The voltage may be applied for up to about 20 minutes, up to about 15 minutes, up to about 10 minutes, up to about 5 minutes, or up to about 3 minutes. The applied voltage may be up to about 25V, up to about 20V, up to about 15V, up to about 10V, up to about 5V, or up to about 3V. Applying an electric current to the system may cause at least a portion of the proteinaceous or hybridization composition (e.g., the secondary antibody or nucleic acid probe and the optional blocking reagent) to migrate from the second carrier matrix to the protein blotting membrane, where the secondary antibody binds to the antigen-bound primary antibody.

When the voltage is terminated, the system may be disassembled and the protein blotting membrane is retrieved and subjected to at least one washing step. The washing steps are typically performed to remove any unbound secondary antibody and thereby increase the signal-to-noise ratio of downstream collected data. The membrane may be immersed in at least 2 ml, at least 5 ml, at least 10 ml, or at least 20 ml of an appropriate buffer (e.g., one of the buffer systems described above and incorporated herein) optionally in the presence of a detergent. Each washing step is typically performed for at least 1 min, at least 2 min, at least 5 min or at least 10 min, though longer or shorter washes are permissible. During a typical 2-step procedure, three 5 minute washes are performed. After the washing steps, a detection step as described above and incorporated herein is performed.

### Kits for Electro-Blotting:

In yet another aspect, provided herein are kits for performing electro-blotting. In one embodiment, a kit may include in at least a first suitable container at least one body of gel matrix that comprises an ion source for electrophoresis and at least one blotting membrane. The body of gel matrix can have a composition as described herein, and preferably includes a buffer ion source. A body of gel matrix and a blotting membrane provided together in a kit can have length and width dimension that are the same or nearly the same, such as within 10%, within 5%, or within 2% of one another in length and width.

In another embodiment, a kit may include in at least a first suitable container at least one body of anodic gel matrix and at least one body of cathodic gel matrix, in which the anodic gel matrix includes at least one anionic buffer compound not present, or present in significantly reduced amounts, in the cathodic gel matrix. As described in previous sections, the anionic buffer compound is preferably a buffer compound with a pKa at or near neutrality. Preferably, both the anode gel matrix and the cathodic gel matrix comprise buffer ion sources, and the cathode compartment includes a buffer compound that is not present (or present in significantly reduced amount) in the anode compartment, in which the cathode buffer compound has a pKa at least about 0.5 log units higher, such as about one log unit higher, than a buffer in the anodic compartment, in which the buffer forms an anion above neutral pH.

In another embodiment, a kit may include in at least a first suitable container at least one body of anodic gel matrix and at least one body of cathodic gel matrix, in which either of both of a cathodic gel matrix or an anodic gel matrix can comprise an ion exchange matrix.

A body of anodic gel matrix and a body of cathodic gel matrix may be provided in a kit in sealed packages. Electro-blotting gel matrix kits can also optionally further include at least one blotting membrane, at least one sheet of filter paper, at least one sponge (such as, e.g., a disposable sponge, and/or at least one electrode. Blotting membranes can be provided juxtaposed with a body of gel matrix, or separately.

In an embodiment, a kit may include in at least a first suitable container a plurality of anodic gel matrix bodies and cathodic gel matrix bodies. In some embodiments, a kit may include from 1 to about 50 anodic gel matrix bodies and cathodic gel matrix bodies. In some embodiments, a kit may include from about 5 to about 20 anodic gel matrix bodies and cathodic gel matrix bodies. In some embodiments, a kit may include from about 8 to about 15 anodic gel matrix bodies and cathodic gel matrix bodies. In some embodiments, a kit may include from about 10 to about 12 anodic gel matrix bodies and cathodic gel matrix bodies.

In another aspect, a kit of the invention provides one or more disposable anodic electrode assemblies and/or one or more disposable cathodic electrode assemblies. In some embodiments, one or more anodic electrode assemblies can include a body of gel including a source of ions and an electrode juxtaposed with a gel matrix. In some embodiments, one or more cathodic electrode assemblies can include a body of gel including a source of ions and an electrode juxtaposed with a gel matrix.

In some embodiments, an anode of an electrode assembly provided in a kit has a surface juxtaposed with an anodic body of gel matrix that contacts at least 50%, at least 60%, more preferably at least 70%, at least 80%, or at least 90% of the side of the anodic gel matrix it is juxtaposed with. In preferred embodiments, an anode of an electrode assembly provided in a kit has a surface juxtaposed with an anodic body of gel matrix that has length and width dimensions that are within 20%, within 10%, within 5%, or within 2% of the length and width dimensions of the side anodic body of gel matrix it is juxtaposed with. In exemplary embodiments, the anode and anodic body of gel matrix are generally rectangular.

In some embodiments, a cathode of an electrode assembly provided in a kit has a surface juxtaposed with a cathodic body of gel matrix that contacts at least 50%, at least 60%, more preferably at least 70%, at least 80%, or at least 90% of the side of the cathodic gel matrix it is juxtaposed with. In preferred embodiments, an cathode of an electrode assembly provided in a kit has a surface juxtaposed with an cathodic body of gel matrix that has length and width dimensions that are within 20%, within 10%, within 5%, or within 2% of the length and width dimensions of the side cathodic body of gel matrix it is juxtaposed with. In exemplary embodiments, the cathode and cathodic body of gel matrix are generally rectangular.

In an embodiment, a kit may include a plurality of anodic assemblies and cathodic assemblies. In some embodiments, a kit may include from 1 to about 50 anodic and cathodic assemblies. In some embodiments, a kit may include from about 5 to about 20 anodic and cathodic assemblies. In some embodiments, a kit may include from about 8 to about 15 anodic and cathodic assemblies. In some embodiments, a kit may include from about 10 to about 12 anodic and cathodic assemblies.

In an embodiment, each anodic assembly and/or each cathodic assembly can be provided in a tray, such as a plastic tray as described below.

The anodic and/or cathodic gel matrix bodies, the anodic and/or cathodic assemblies, or the anodes and/or the cathodes can be enclosed within a sealed package together, or separately. Furthermore, multiple anodic and/or cathodic assemblies can be enclosed together in packaging. In certain embodiments, a plurality of anodic assemblies or anodic gel matrices may be referred to collectively as bottom consumables, and a plurality of cathodic assemblies may be referred to as top consumables.

In some aspects, an electro-blotting kit includes one or more disposable anodic assemblies and one or more disposable cathodic assemblies. In some aspects, an electro-blotting kit includes one or more disposable anodic electrode assemblies and at least one body of cathodic gel matrix. The kits may optionally include one or more carrier matrices, sheets of filter paper, or sponges.

In an embodiment, a kit may include one or more carrier matrices. The carrier matrices may be in the form of sheets configured such that the sheets are juxtaposable with the anodic assembly, the cathodic assembly, or the anodic and the cathodic assemblies. The properties and composition of carrier sheets suitable for inclusion in a kit as presently contemplated are described above and incorporated herein.

In an embodiment, the dimensions of the carrier matrix sheets provided with a kit may be at least as large as or smaller than the dimension of the anodic assembly and the cathodic assemblies. In an embodiment, one side of the carrier matrix sheets may be in the range of about 1 cm to about 50 cm, about 5 cm to about 20 cm, about 8 cm to about 15 cm or about 10 cm to about 12 cm. The other side of the carrier matrix sheet may be the range of about 1 cm to about 50 cm, about 5 cm to about 20 cm, about 8 cm to about 15 cm or about 10 cm to about 12 cm. The thickness of carrier matrix sheets provided with a kit according to some embodiments may be less than about 5 mm, less than about 4 mm, less than about 3 mm, less than about 2 mm, less than about 1 mm, less than about 0.5 mm or less than about 0.25 mm.

In an embodiment, each kit may be supplied to an end user with at least one carrier matrix. Typically, a plurality of carrier matrices will be supplied in a kit as presently contemplated. In some embodiments, a kit may include between 1 to about 50 carrier matrix sheets. In some embodiments, a kit may include between about 5 to about 25 carrier matrix sheets. In some embodiments, a kit may include between about 10 to about 15 carrier matrix sheets. In some embodiments, a kit may include between about 10 to about 12 carrier matrix sheets.

In some embodiment, carrier matrix sheets may be packaged separately from the anodic assembly and the cathodic assembly. A group of carrier matrices may be supplied as a unit packaged together in a single package. In an embodiment, each carrier matrix may be individually packaged and each individually packaged carrier matrix may further be packaged as a unit with a plurality of other individually packaged carrier matrices. In an embodiment, one or more carrier matrices may be packaged together with an anodic assembly. In an embodiment, one or more carrier matrices may be packaged together with a cathodic assembly.

In some embodiments, a kit may also separately provide one or more electrodes. Electrodes can be provided, for example, in a sealed container that may, in certain embodiments, also include a dessicant or an anti-corrosive agent. The electrodes can be packaged in liquid or gel, such as an alcohol or a solution or gel comprising one or more preservatives, reducing agents, or anti-corrosives. Kits providing electrodes, such as disposable electrodes, can also include one or more gel matrices, one or more blotting membranes, or one or more sheets of filter paper.

The anodic and/or the cathodic electrode assemblies of the kit, optionally including the one or more carrier matrices, may be individually wrapped in a suitable gas and water impermeable wrapper (or any other type of suitable container), as is known in the art, in order to enable storage of the electrode assemblies for extended periods of time without drying. For example, the wrapper or container may be made from a suitable thin, water and gas impermeable plastic or polymer based sheet or foil, and may be sealed after packaging of the electrode therein using any suitable wrapper sealing method known in the art (such as, but not limited to gluing or contact heat sealing, or the like). Blotting membranes, when provided in kits, can be provided in separate wrapping, or together within a package that includes an electrode assembly.

Thus it will be appreciated by those skilled in the art that various different combinations and sub-combinations of the various different electrode assemblies disclosed hereinabove may be combined to form many different types of kits. Suck kits may or may not include different stains as is known in the art and/or stain releasing metals (such as, for example anodic silver metal containing electrode assemblies, as disclosed hereinabove, depending on the application. Similarly the gel concentrations and compositions and the degree of cross linking may be varied to in accordance with the blotted species.

Furthermore, the dimensions of the various possible kit parts such as the different types of electrode assemblies and/or blotting membranes may be modified or adapted for use with the particular dimensions of the gel to be blotted, as will be readily apparent to the skilled artisan.

It is also possible to include in such wrappers a suitable shallow open tray (not shown) made of plastic or other suitable material. The tray may have dimensions suitable for receiving the carrier matrix or the electrode assembly therein to protect the components from mechanical damage during handling and to facilitate the handling and manipulation of the components after the wrapper is opened before use. Alternatively, the tray may be used to hold the carrier matrix while the blotting buffers are applied thereto, as described in detail above and incorporated herein. A tray may also be sealed over the top with fluid-impermeable plastic or foil (or foil-backed plastic), and the top sheet of plastic or foil can be removed to expose the electrode assembly for use. An electrode assembly (such as, for example, a cathode assembly) can be removed from the tray for use, or the electrode assembly can remain positioned within the tray during electro-blotting.

The holding tray may be a rectangular tray to accommodate the shape of an electrode assembly or carrier matrix. However, the holding tray may be made in other suitable shapes, depending, inter alia, on the shape of the electrode assembly (which in turn may vary in shape depending, inter alia, on the application). The holding tray may preferably be made from an inexpensive rigid or semi-rigid plastic or polymer such as, but not limited to, polyvinylchloride (PVC). It is, however, noted that any other suitable material(s) may be used for forming the holding tray.

The holding tray may also function as a stabilizer in the process of forming the blotting assembly prior to performing the electro-blotting transfer.

In one embodiment, a kit may include one or more containers of a diluent suitable for use with the system described above. The diluent may be used to prepare a proteinaceous composition or a hybridization composition as described above. A diluent may include a physiologically acceptable aqueous solution having a pH in the range of about 4 to about 9, or from about 5 to about 8, or from about 6 to about 7.5, and typically having at least one buffering agent such as, e.g., phosphate buffer, bicarbonate, TAPS, Bicine, Tris, Bis-Tris, Tricine, HEPES, TES, MOPS, PIPES, Cacodylate, MES, acetate, ADA, ACES, cholamine, BES, acetamidoglycine or glycinaide present therein. Exemplary buffers suitable for use as diluents may include, though are not limited to, e.g., PBS, Hank's solution, TBS, TE, TEN, or the like. Optionally, a diluent may include a detergent. Suitable detergents may include non-ionic, non-denaturing detergents such as, e.g., Triton X-100, Triton X-114, NP-40, Brij-35, Brij 58, Tween-20, Tween-80, octyl glucoside and octylthio glucoside. A diluent may contain from about 0.01 vol % to about 5 vol. %, from about 0.05 vol % to about 2 vol. %, from about 0.1 vol % to about 1.5 vol. %, or from about 0.5 vol % to about 1 vol. % of a non-ionic non-denaturing detergent.

In some embodiments, a diluent may be supplied at full strength (i.e., 1× strength) or may be supplied as a concentrated solution that facilitates storage and shipping thereof. A concentrated diluent may be diluted by the user. Concentrated diluents may be supplied as up to about 50×, up to about 25×, up to about 20×, up to about 10×, or to about 5× or up to about 2× strength.

In some embodiments, a diluent may be supplied to a user in one or more plastic, PVC or glass bottles supplied with the kit. Each kit may include between 1 to 10 bottles of a diluent, between 1-5 bottles of a diluent, or between 1-2 bottles of a diluent. Each bottle of diluent may contained up to 5 L, up to 4 L, up to 3 L, up to 2 L, up to 1 L, up to 500 ml, or up to 100 ml of a diluent.

In some embodiments, a kit may include a suitable blocking reagent. A blocking reagent may be supplied dissolved or dispersed in the diluent or may be supplied separately from the diluent. A blocking reagent may include, by way of non-limiting example, whole serum, fractionated serum, bovine serum albumin, casein, soy protein, non-fat milk, gelatin, fish serum, goat immunoglobulin, rabbit immunoglobulin, mouse immunoglobulin, rat immunoglobulin, horse immunoglobulin, human immunoglobulin, pig immunoglobulin, chicken immunoglobulin or synthetic blocking reagents, such as those that may be obtained commercially form, e.g., BioFX Laboratories, Kem-En-Tec Diagnostics or GeneWay Biotech. A variety of commercially available pre-prepared blocking reagents are available in the art, all of which may be supplied with a kit as described herein. Such commercially available blocking reagents include, though are not limited to, e.g., WesternBreeze, I-BLOCK, Blocklt, PerfectBlock, Synthetic Blocking Buffer (BioFX Labs), Gelantis BetterBlock, SeaBlock, Starting Block and Protein-Free Blocking Buffer (Pierce). In embodiments where the blocking reagent is supplied dissolved or dispersed in the diluent, the amount of the blocking reagent present may be in the range of about 0.1 wt. % to about 50 wt. %, about 1 wt. % to about 40 wt. %, about 2.5 wt. % to about 25 wt. %, about 5 wt. % to about 15 wt. % or about 10 wt %. In an embodiment, the amount of a blocking reagent present in an blotting buffer may be up to about 75 mg/ml, up to about 50 mg/ml, up to about 40 mg/ml, up to about 30 mg/ml, up to about 20 mg/ml, up to about 15 mg/ml, up to about 10 mg/ml up to about 5 mg/ml, up to about 2.5 mg/ml, up to about 1 mg/ml, up to about 0.5 mg/ml, up to about 0.25 mg/ml or up to about 0.1 mg/ml.

In some embodiments, a kit may include a hybridization reagent or hybridization buffer suitable for use in performing nucleic acid hybridization experiments. The term pre-hybridization buffer, or more colloquially, “pre-hyb buffer”, may be used interchangeably with “hybridization reagent” or “hybridization buffer”. A variety of pre-hybridization buffers are well-known to those having ordinary skill in the art, and any may be used without limitation.

In some embodiments, a kit may include one or more containers of a wash buffer. Any suitable wash buffer known to those skilled in the art may be supplied with a kit in accordance with the presently described embodiments. In some embodiments, a suitable wash buffer may be the same as the diluent. In some embodiments, the wash buffer may be the diluent lacking one or more components thereof. In some embodiments, the wash buffer may be the diluent lacking a blocking reagent. By way of non-limiting example, a wash buffer may include any of the following aqueous buffered solutions; phosphate buffer (PBS), bicarbonate, TAPS, Bicine, Tris, Bis-Tris, Tricine, HEPES, TES, MOPS, PIPES, Cacodylate, MES, acetate, ADA, ACES, cholamine, BES, acetamidoglycine or glycinaide present therein. Exemplary buffers suitable for use as wash buffers may include, though are not limited to, e.g., PBS, Hank's solution, TBS, TE, TEN, or the like. Optionally, a wash buffer may include a detergent. Suitable detergents may include non-ionic, non-denaturing detergents such as, e.g., Triton X-100, Triton X-114, NP-40, Brij-35, Brij 58, Tween-20, Tween-80, octyl glucoside and octylthio glucoside. A diluent may contain from about 0.01 vol. % to about 5 vol. %, from about 0.05 vol. % to about 2 vol. %, from about 0.1 vol. % to about 1.5 vol. %, or from about 0.5 vol. % to about 1 vol. % of a non-ionic non-denaturing detergent.

In some embodiments, a wash buffer may be supplied at full strength (i.e., 1× strength) or may be supplied as a concentrated solution that facilitates storage and shipping thereof. A concentrated wash buffer may be diluted by the user. Concentrated wash buffers may be supplied as up to about 50×, up to about 25×, up to about 20×, up to about 10×, up to about 5× or up to about 2× strength.

In some embodiments, a wash buffer may be supplied to a user in one or more plastic, PVC or glass bottles supplied with the kit. Each kit may include between 1 to 10 bottles of a wash buffer, between 1-5 bottles of a wash buffer, or between 1-2 bottles of a wash buffer. Each bottle of diluent may contained up to 5 L, up to 4 L, up to 3 L, up to 2 L, up to 1 L, up to 500 ml, or up to 100 ml of a diluent.

In some embodiments, a kit may include one or more primary antibodies supplied in a suitable container. The kit may include up to 1 ml, up to 750 pt, up to 500 pt, up to 250 pt, up to 200 pt, up to 150 pt, up to 100 pt or up to 50 pt of a primary antibody. The primary antibody may be dispersed in a suitable aqueous storage medium. The primary antibody may be adapted to be stored at room temperature, in a refrigerated environment or in a freezer.

In certain embodiments, a primary antibody supplied with a kit may be a polyclonal antibody or a monoclonal antibody. A monoclonal antibody may be raised in mouse or in rat. A monoclonal antibody may be IgG (IgG1, IgG2a, IgG2b, IgG3), IgM, IgA, IgD and IgE subclasses. A polyclonal antibody may be raised in rabbit, mouse, rat, hamster, sheep, goat, horse, donkey or chicken. In an embodiment, an antibody may be derived from human serum. A human antibody may be at least partially or fully purified. Methods of preparing and purifying antibodies are widely known in the art. General guidance in the production and use of various antibody preparations may be found, for example in the reference texts Harlow et al., 1989, Antibodies: A Laboratory Manual, Cold Spring Harbor, N.Y., Harlow et al., 1999, Using Antibodies: A Laboratory Manual, Cold Spring Harbor Laboratory Press, NY, and Harlow, et al., 1988, In: Antibodies, A Laboratory Manual, Cold Spring Harbor, N.Y., all of which are hereby expressly incorporated by reference.

In some embodiments, a primary antibody supplied with a kit may be a loading control antibody. Exemplary though non-limiting loading control antibodies that may be supplied with the presently described systems and methods may include antibodies directed against actin, tubulin, histone, vimentin, lamin, GAPDH, VDAC1, COXIV, hsp-70, hsp-90 or TBP. Other loading control antibodies may also be included such as will be readily apparent to a practitioner having ordinary skill in the art.

In some embodiments, a kit may include one or more secondary antibodies supplied in a suitable container. The kit may include up to 1 ml, up to 750 pt, up to 500 pt, up to 250 pt, up to 200 pt, up to 150 pt, up to 100 pt or up to 50 pt of a secondary antibody. The secondary antibody may be dispersed in a suitable aqueous storage medium. The secondary antibody may be adapted to be stored at room temperature, in a refrigerated environment or in a freezer.

A secondary antibody provided as a component of a kit as presently embodied may be raised in rabbit, mouse, rat, hamster, pig, sheep, goat, horse, donkey, turkey or chicken. The secondary antibody may be at least partially affinity purified. The secondary antibody may be directed against mouse IgG, mouse IgA, mouse IgM, rat IgG, rat IgA, rat IgM, rabbit IgG, rabbit IgA, rabbit IgM, hamster IgG, hamster IgA, hamster IgM, goat IgG, goat IgA, goat IgM, horse IgG, horse IgA, horse IgM, sheep IgG, sheep IgA, sheep IgM, donkey IgG, donkey IgA, donkey IgM, chicken IgG, chicken IgA, chicken IgM, chicken IgY, human IgG, human IgA, or human IgM. A secondary antibody may be coupled to one or more detection molecules such as, by way of example, alkaline phosphatase, peroxidase, biotin, a fluorophore or Qdot nanocrystals.

In some embodiments, a kit may be provided having one or more bottom consumables, one or more top consumables and one or more carrier matrices packaged together in a first kit container. The first kit container may be stored at room temperature or in a refrigerated environment. The kit may also be provided having one or more containers of diluent, one or more containers of wash buffer, one or more containers of primary antibody, one or more containers of secondary antibody, or one or more containers of developing reagent packaged together in at least a second kit container. The second kit container may be stored at room temperature or in a refrigerated environment. In some embodiments, at least a subset of the contents of the second kit container (such as, e.g., the primary antibodies or the secondary antibodies) may be stored in a freezer.

In some embodiments, a kit may include one or more nucleic acid probes. The nucleic acid probes may be oligonucleotides of full or partial length cDNAs, or may be single or double-stranded. In an embodiment, a nucleic acid probe may be labeled or unlabeled. In some embodiments, a kit may include one or more reagent for labeling a nucleic acid probe. Certain nucleic acid probes may be provided to provide internal experimental control reagents. Such probes may include, e.g., DNA or RNA probes to tubulin, actin, vimentin, GAPDH, etc.

In some embodiments, a kit may further include instructions on the use and/or storage of each component of the kit. The instructions may direct or instruct the user how to perform one or more aspects of an electro-blotting procedure. The instructions may be provided as a hard copy supplied with the kit at the time of its delivery to the customer. Alternatively, instructions may be provided to the end user by way of one or more electronic communication means (e.g., e-mail or the website of the company providing the kit).

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that one or more changes can be made in the specific embodiments which are disclosed and still obtain a like or similar result without departing from the spirit and scope of the embodiments set forth herein.

### Example 1

### Several Blotting Reagent Possess an Inherent Negative Charge

Without being bound by any particular theory or mechanism of action, because the presently described system and method relies in part on the electrophoretic transfer of blotting reagents from a carrier matrix to molecules immobilized on a solid support, reagents (such as, e.g., primary antibodies, secondary antibodies, blocking reagents, and the like), an experiment was performed to demonstrate that such reagents are able to undergo electrophoretic transfer under non-denaturing (i.e., in the absence of SDS) conditions. FIG. 3 is an image demonstrating the inherent negative charge at neutral pH of various reagents used with an electro-blotting detection system according to an embodiment. Samples were resolved on a native 1.2% E-GEL® clear (Invitrogen Corp, Carlsbad, Calif.) and the gel was stained with Coomassie to visualize resolved proteins. Samples are as follows: lane 1, WESTERNBREEZE® Blocking Solution; lane 2, mouse anti-actin monoclonal antibody; lane 3, mouse anti-tubulin monoclonal antibody; lane 4, goat anti-rabbit secondary antibody coupled to alkaline phosphatase; lane 5, goat anti-mouse secondary antibody coupled to alkaline phosphatase. These results demonstrate that blotting reagents possess an inherent negative charge

### Example 2

### Comparison of Conventional Vs. Electro-Blotting Procedures

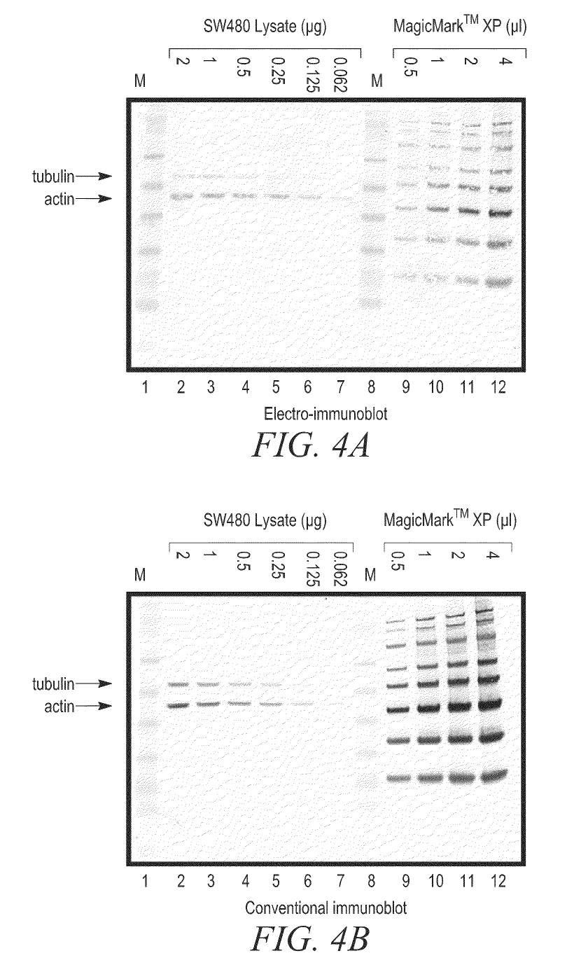

FIGS. 4A and 4B show the results obtained after performing a blotting procedure on SW480 cell lysate to detect tubulin and actin according to an embodiment of the presently described electro-blotting system and methods (FIG. 4A) or using conventional blotting techniques (FIG. 4B) or.

SW-480 cell lysate was obtained commercially from Prosci incorporated, CA. Serial two-fold dilutions of the lysate (2 μg-62 ng; lanes 2-7 of FIGS. 4A and 4B) were resolved along with the indicated volume of MAGICMARK™ molecular weight protein markers (lanes 9-12) on a NUPAGE® Novex 4-12% Bis-Tris Gel (Invitrogen Corp.) according to manufacturer instructions. Resolved proteins were transferred to a Nitrocellulose (NC) protein blotting membrane using the IBLOT™ Dry Blotting System (Invitrogen Corporation, Carlsbad, Calif.) on P3 for 7 minutes or using the NOVEX® semi wet blot module at 30V for 1 hr. The membrane was blocked using the WESTERNBREEZE® kit blocking solution for 30 minutes at room temperature on a rotational shaker and washed twice for 5 minutes using WESTERNBREEZE® washing solutions.

In FIG. 4B, membranes were processed for blotting according to the WESTERNBREEZE® Chromogenic Detection Kit instructions. Blotting was performed using 1:5000 and 1:10,000 dilutions of anti-actin and anti-tubulin monoclonal antibodies, respectively, in WESTERNBREEZE® diluent for 1 hour at room temperature on a rotational shaker. The blotting solution was removed and the membrane was washed three times for 5 minutes each. Next, the membranes were incubated for 30 minutes with anti-mouse secondary antibody alkaline phosphatase (AP) conjugate of the WESTERNBREEZE® kit on a rotational shaker. The second blotting solution was removed and the membranes were washed 3 times for 5 minutes each and developed chromogenically according to manufacturer instructions.

In FIG. 4A, electro-blotting of SW480 cell lysate immobilized on NC membrane using mouse anti-tubulin and anti-actin primary antibody was performed as follows: following transfer, the membrane was blocked as described above and was processed for electro-immunoblot using the IBLOT™ system. The membrane was placed on a mini IBLOT™ bottom stack. 3.5 ml of a solution containing the primary (1:2500) and secondary antibodies (anti-mouse conjugated AP, 1:5000) was applied to a Whatman filter paper carrier matrix using a pipette. The matrix was then placed on top of the membrane and a blotting roller was used to remove air bubbles. The top stack was then placed on top of the matrix and the lid of the IBLOT™ apparatus was closed. The IBLOT™ was set to a program of P5 for 7 minutes. When the program run was complete the membrane was removed, washed three times in WESTERNBREEZE® washing solution and developed as discussed above.

### Example 3

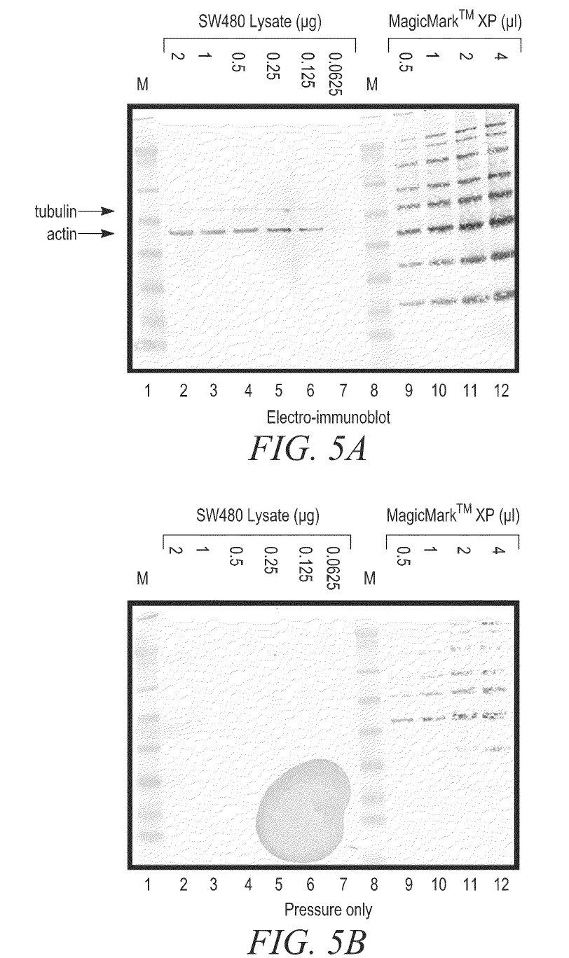

FIGS. 5A-B show the results of an experiment demonstrating that the electrical field has a major contribution to the electro-blotting process, but the pressure has also some additive effect. FIG. 5A shows the results obtained after performing an electro-blotting procedure. The electro-blotting procedure was performed as described above in EXAMPLE 2 for FIG. 4A. FIG. 5B shows the results obtained when the procedure is repeated without running program P5 on the IBLOT™ apparatus.

### Example 4

### Comparison of Filter Paper Vs. Polyester/Polyamide Microfiber Carrier Matrices

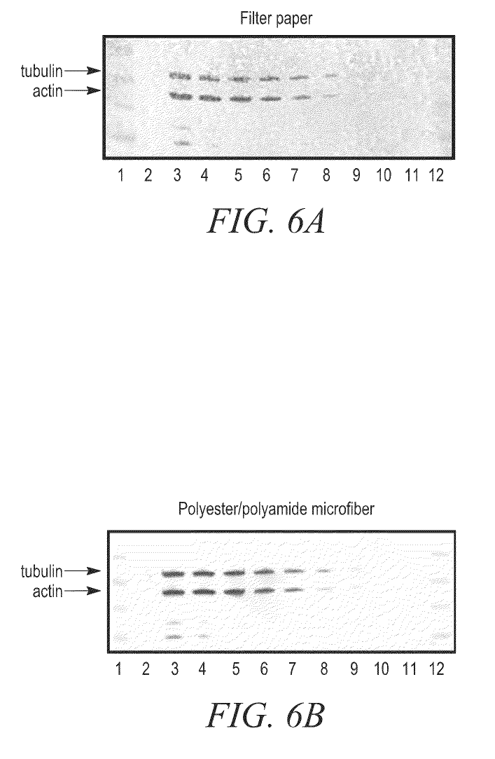

FIGS. 6A-B show a comparison between the results obtained using different carrier matrices. Protein molecular weight standard (lane 1) and SW480 serial 2-fold dilutions of cell lysate (lanes 3-10) were resolved by SDS-PAGE and transferred to NC membranes as described above in Example 2. In FIG. 6A, an electro-blotting experiment was conducted essentially as described above with regard to FIG. 4A. In FIG. 6B, an electro-blotting experiment was conducted essentially as described above with regard to FIG. 4A except that the filter paper carrier matrix was replaced with a sheet of polyester/polyamide microfiber (obtained commercially from Sadovsky Houshold products, Ltd. Ashdod, Israel).

### Conventional Blotting Vs. Electro-Blotting Using Different Conjugates, Detection Methods, Transfer Methods and Membranes (Examples 5-8)

### Example 5

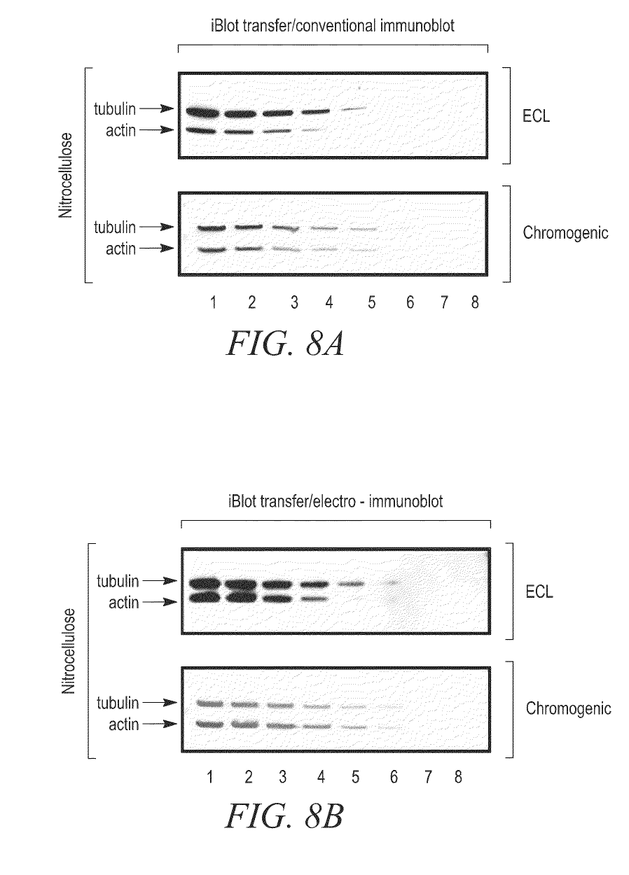

FIGS. 8A-B show a comparison between the results obtained using chemiluminescent or chromogenic detection methods for blotting experiments performed using conventional methods (FIG. 8A) or electro-blotting methods (FIG. 8B). Serial 2-fold dilutions SW480 of cell lysate (lanes 1-8) were resolved by SDS-PAGE and transferred to NC membranes as described above in Example 2. In FIG. 8A, conventional blocking and blotting steps were performed essentially as described above. In FIG. 8B, electro-blotting was conducted essentially as described above in Example 2, except that the filter paper carrier matrix was replaced with a sheet of polyester/polyamide microfiber as described in Example 4, and the IBLOT™ was set to a program of 5V for 5 minutes. When the program run was complete the membrane was removed, washed three times in WESTERNBREEZE® washing solution and developed as described above and according to manufacturer's instructions. Results shown in FIGS. 8A and 8B include those obtained with the use of anti-mouse HRP-coupled secondary antibody developed using ECL (upper panel) and anti-mouse AP-coupled secondary antibody developed using chromogenic methods (lower panel).

### Example 6

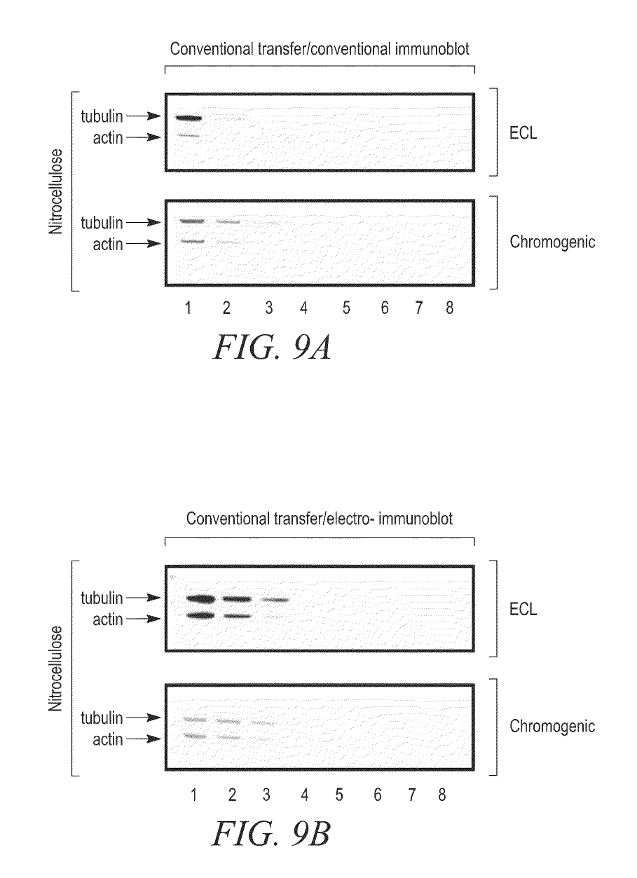

FIGS. 9A-B show results obtained using electro-blotting methods essentially as described in Example 5, except that transfer of proteins from the electrophoresis gel to the NC membrane was achieved using conventional “wet” transfer methods.

### Example 7

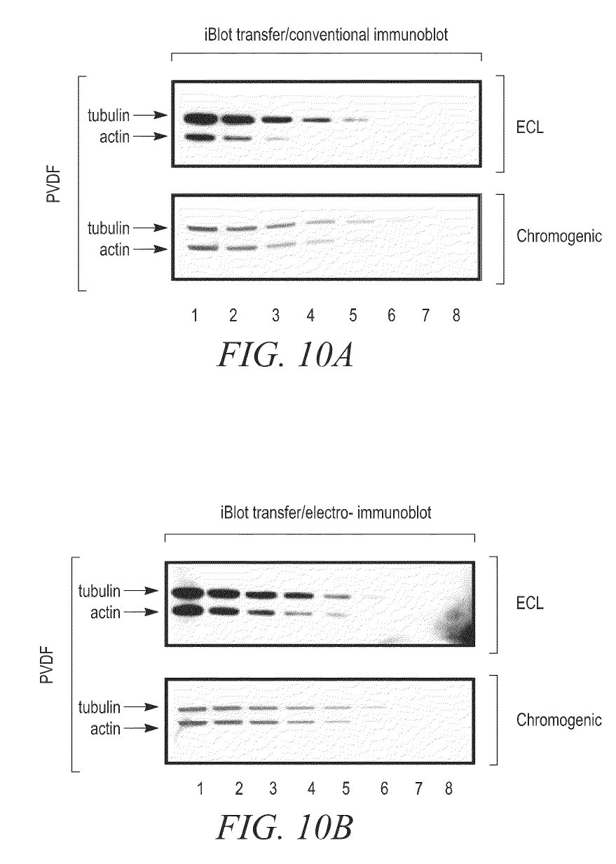

FIGS. 10A-B show results obtained using electro-blotting methods essentially as described in Example 5, except that the NC protein blotting membrane is replaced by a PVDF protein blotting membrane.

### Example 8

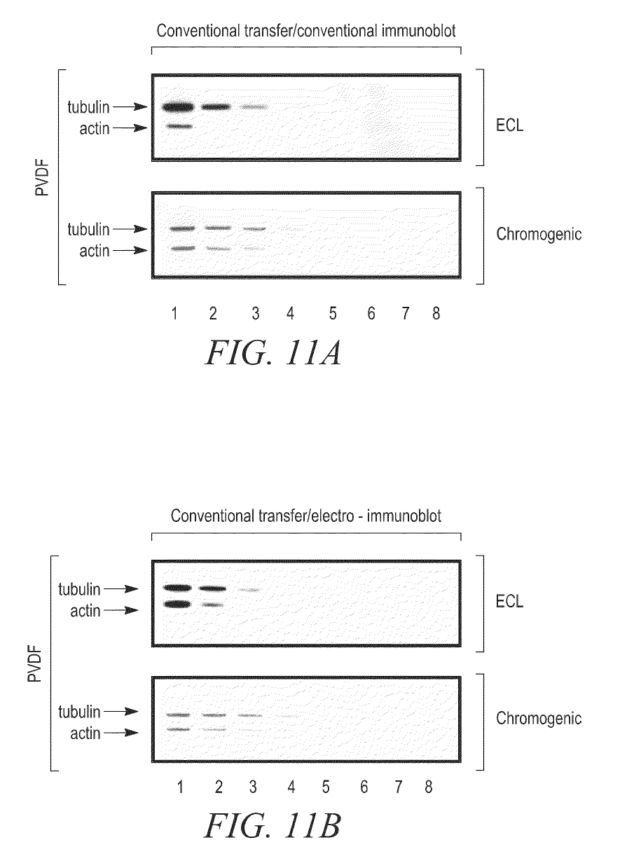

FIGS. 11A-B shows results obtained using electro-blotting methods essentially as described in Example 6, except that the NC protein blotting membrane is replaced by a PVDF protein blotting membrane.

### Conventional Blotting Vs. One Step Electro-Blotting

### Example 9

This example discloses a simplified method wherein the blocking reagent, primary antibody, and secondary antibody are included onto an immunoblot membrane using SW480 cell lysate and anti-tubulin and anti-actin antibodies

SW-480 cell lysate samples (1 μg-62.5 ng in two-fold dilutions) were loaded on NUPAGE® Novex 4-12% Bis-Tris Gel. The gel was run for 37 minutes to separate the protein samples and the separated proteins were transferred to NC membrane using an IBLOT™, at 20V for 7 minutes, as discussed in Example 2. A control membrane was treated and developed according to the conventional methods of Example 2.

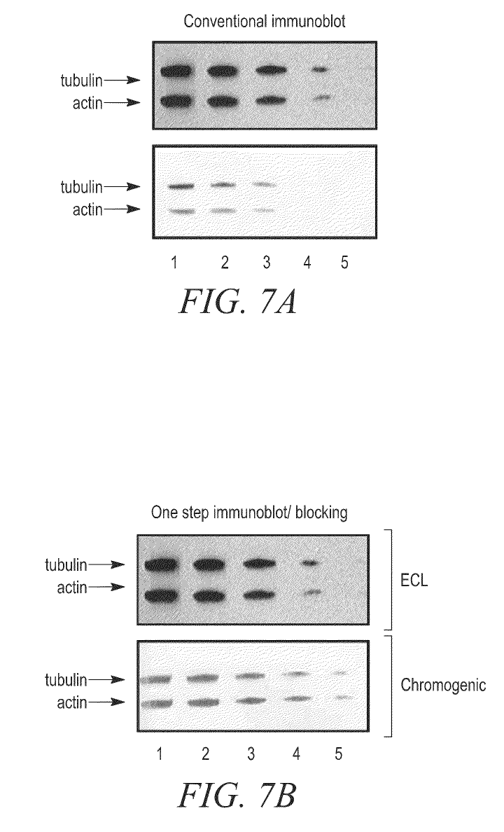

The unblocked membrane was processed according to a method of the invention as follows. The unblocked membrane was placed on an IBLOT™ bottom stack. The primary and secondary antibodies in this exemplary procedure are diluted in a blocking solution of 25% Synthetic-Blocking Buffer (Catalog No. STSB-0100-01 available from BioFX, Owings Mills, Md.), 1% Casein, 200 mM NaCl and 10 mM Bis-Tris. The diluted solution, 3.5 ml, containing the primary (1:2500) and secondary antibodies (anti-mouse Conjugated HRP or AP, 1:5000) is applied to a polyester/polyamide microfiber matrix, as described in Example 4. The IBLOT™ in this case was set to a program of 5V for 3 minutes. When the program run was complete the membrane was removed, washed three times in WESTERNBREEZE® washing solution and developed as discussed in Example 2. The results, in comparison to a control membrane treated and developed according to the methods of Example 2 are shown in FIGS. 7B and 7A respectively.

### Conventional Blotting Vs. Two Step Electro-Blotting (Examples 10-11)

### Example 10

E. coli cell lysate samples purchased from Promega (1.25 μg-78.5 ng in double dilutions) were loaded on NUPAGE® Novex 4-12% Bis-Tris Gel. The gel was run for 37 minutes to separate the protein samples and the separated proteins were transferred to NC membrane using an IBLOT™, at 20V for 7 minutes. The membrane was then blocked using the WESTERNBREEZE® kit blocking solution for 30 minutes on a rotational shaker and washed twice for 5 minutes using WESTERNBREEZE® washing solutions

The membrane was processed for blotting according to the WESTERNBREEZE® Chemiluminescent Detection Kit instructions. Blotting was performed using 1:5000 dilutions of rabbit anti E. coli polyclonal antibodies, in WESTERNBREEZE® diluent for 1 hour on rotational shaker. The blotting solution was removed and the membrane was washed three times for 5 minutes each. Next, the membrane was incubated for 30 minutes with an anti-rabbit secondary antibody alkaline phosphatase based conjugate solution of the WESTRNBREEZE® kit on a rotational shaker. The second blotting solution was removed and the membranes were washed 3 times for 5 minutes each and developed by chemiluminescent detection.

### Example 11

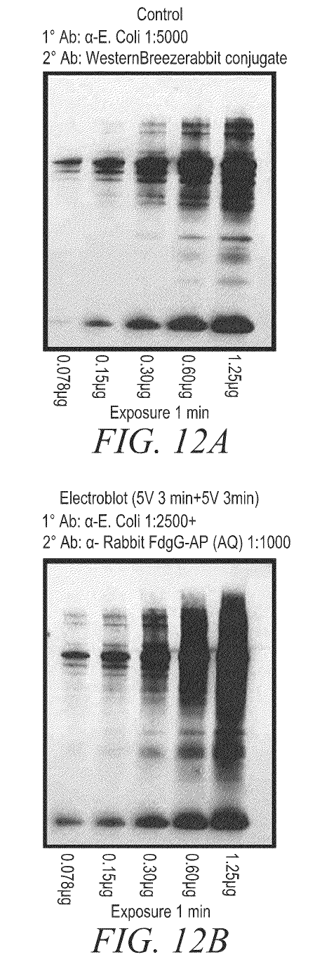

This example discloses a simplified method wherein the blocking reagent and primary antibody are included on to an immunoblot membrane in one step and then the secondary antibody (in blocking solution) is included onto the immunoblot membrane in a different step using E. coli cell lysate and anti-E. coli antibody. E. coli cell lysate samples purchased from Promega (1.25 ug-78.5 ng in double dilutions) were loaded on NUPAGE® Novex 4-12% Bis-Tris Gel. The gel was run for 37 minutes to separate the protein samples and the separated proteins were transferred to NC membrane using an IBLOT™, at 20V for 7 minutes, as discussed in Example 10. The unblocked membrane was processed according to a method of the invention as follows. The unblocked membrane was placed on a mini IBLOT™ bottom stack. Separate solutions are prepared by diluting the primary antibody or the secondary antibodies in a blocking solution of 30% Synthetic-Blocking Buffer (Catalog No. STSB-0100-01 available from BioFX, Owings Mills, Md., 0.3% Soy isolate, 200 mM NaCl and 10 mM Bis-Tris. The diluted solution, 3.5 ml, containing the primary (1:2500) antibody was applied to a polyester/polyamide microfiber matrix, as described in Example 4. The IBLOT™ in this case was set to a program of 5V for 3 minutes. When the program run was complete the carrier matrix was removed and was replaced with a second carrier matrix on which 3.5 ml of the above mentioned diluent solution containing the secondary antibody (goat anti-rabbit Conjugated AP, 1:1000). The IBLOT™ in this case was set to a program of 5V for 3 minutes. When the program run was complete the membrane was removed, washed three times in WesternBreeze® washing solution and developed as discussed in Example 10. The results, in comparison to the control membrane described in Example 10 are shown in FIGS. 12B and 12A respectively.

### Example 12

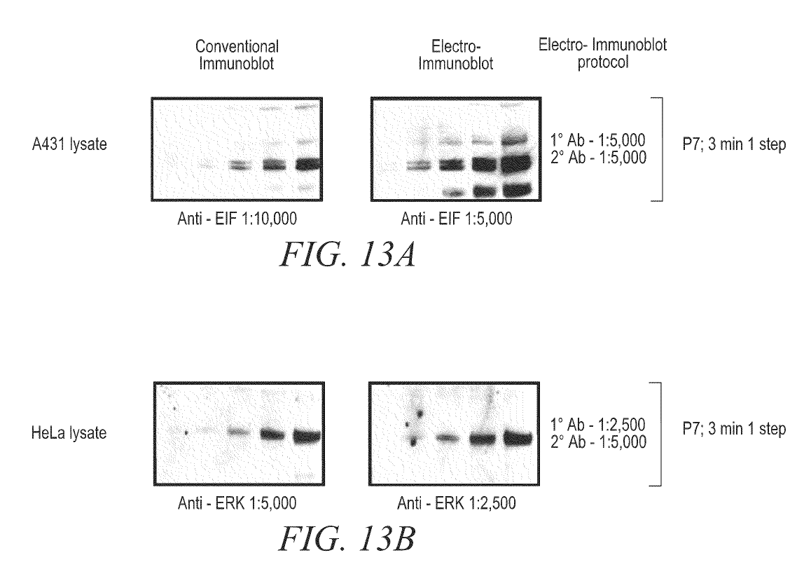

The results depicted in FIG. 13A were obtained essentially as described in EXAMPLE 2 with the following exceptions: 2 μg-62 ng of A431 cell lysate was loaded on two identical NUPAGE® Novex 4-12% Bis-Tris Gel (Invitrogen Corp.) and the proteins were resolved according to manufacturer instructions. Resolved proteins were transferred to Nitrocellulose (NC) protein blotting membranes using the IBLOT™ Dry Blotting System (Invitrogen Corporation, Carlsbad, Calif.) running a program of P3 for 7 minutes. The membranes were blocked using the WesternBreeze® kit blocking solution for 30 minutes at room temperature on a rotational shaker and washed twice for 5 minutes using WesternBreeze® washing solutions.

One of the membranes (depicted in the left hand panel of FIG. 13A) was subjected to conventional Western blotting. Blotting was performed using 1:10,000 dilution of monoclonal anti-Elf antibody in WESTERNBREEZE® diluent for 1 hour at room temperature on a rotational shaker. The blotting solution was removed and the membrane was washed three times for 5 minutes each. Next, the membrane was incubated for 30 minutes with anti-mouse secondary antibody peroxidase conjugate of the WESTERNBREEZE® kit on a rotational shaker. The second blotting solution was removed and the membrane was washed 3 times for 5 minutes each and developed using ECL reagent according to manufacturer instructions.

The other membrane (depicted on the right hand panel of FIG. 13A) was subjected to electro-blotting as follows: following transfer of the A431 lysate to the NC membrane, the membrane was blocked as described above and was processed for electro-immunoblot using the IBLOT™ system. The membrane was placed on a mini IBLOT™ bottom stack. 3.5 ml of a solution containing the primary (1:5,000 mouse anti-Elf) and secondary antibodies (anti-mouse conjugated HRP, 1:5,000) was applied to a matrix of polyester/polyamide microfiber (obtained commercially from Sadovsky Houshold products, Ltd. Ashdod, Israel. The matrix was then placed on top of the membrane and a blotting roller was used to remove air bubbles. The top stack was then placed on top of the matrix and the lid of the IBLOT™ apparatus was closed. The IBLOT™ was set to a program of P7 for 3 minutes. When the program run was complete the membrane was removed, washed three times in WESTERNBREEZE® washing solution and developed using enhanced chemiluminescence as described above.

The results shown in FIG. 13B were obtained essentially as described in FIG. 13A, with the following exception: The A431 lysate was replaced with HeLa cell lysate. For the conventional immunoblot (shown in the left hand panel), the primary antibody was replaced with a 1:5,000 dilution of mouse anti-ERK. The secondary antibody, and the dilution that was used, remained the same. For the electro-immunoblot (shown in the right-hand panel), the dilution of the primary antibody was 1:2.500, and the dilution of the secondary antibody was 1:5.000.

### Example 13

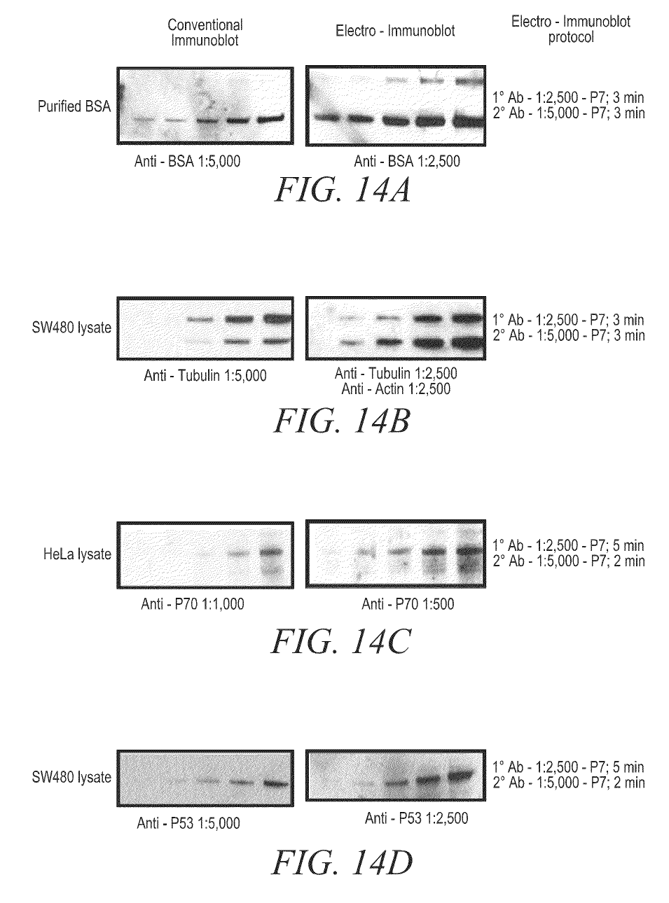

The experiments shown in FIGS. 14A to 14D were performed essentially as described in EXAMPLE 12 with the following exceptions: In FIG. 14A, purified bovine serum albumin (BSA) was resolved in a NUPAGE® Novex 4-12% Bis-Tris Gel. In the conventional immunoblot (shown in the left-hand panel of FIG. 14A), the dilution of the mouse anti-BSA primary antibody was 1:5,000.

For the electro-immunoblot, shown in the right-hand panel of FIG. 14A, 3.5 ml of a 1:2,500 dilution of mouse anti-BSA in antibody in WESTERNBREEZE® diluent was absorbed on the polyester/polyamide microfiber matrix. The IBLOT™ apparatus was set to program P7 for 3 minutes. The matrix was removed from the apparatus, and 3.5 ml of a 1:5,000 dilution of anti-mouse conjugated HRP antibody in WESTERNBREEZE® diluent was absorbed on a second polyester/polyamide microfiber matrix, which was placed over the NC membrane. The IBLOT™ apparatus was closed and run at P7 for another 3 minutes. When the program run was complete the membrane was removed, washed three times in WESTERNBREEZE® washing solution and developed using enhanced chemiluminescence as described above.

In FIG. 14B, SW480 cellular lysate was resolved in a NUPAGE® Novex 4-12% Bis-Tris Gel. In the conventional immunoblot (shown in the left-hand panel of FIG. 14B), the dilution of the rabbit anti-tubulin antibody was 1:5,000. For the secondary antibody, a 1:5,000 dilution of mouse anti rabbit conjugated HRP antibody in WESTERNBREEZE® diluent was used.

For the electro-immunoblot, shown in the right-hand panel of FIG. 14B, 3.5 ml of a 1:2,500 dilution of rabbit anti-tubulin antibody in WESTERNBREEZE® diluent was absorbed on the polyester/polyamide microfiber matrix. The IBLOT™ apparatus was set to program P7 for 3 minutes. The matrix was removed from the apparatus, and 3.5 ml of a 1:5,000 dilution of mouse anti-rabbit conjugated HRP antibody in WESTERNBREEZE® diluent was absorbed on a second polyester/polyamide microfiber matrix, which was placed over the NC membrane. The IBLOT™ apparatus was closed and run at P7 for another 3 minutes. When the program run was complete the membrane was removed, washed three times in WESTERNBREEZE® washing solution and developed using enhanced chemiluminescence as described above.

In FIG. 14C, HeLa cellular lysate was resolved in a NUPAGE® Novex 4-12% Bis-Tris gel. In the conventional immunoblot (shown in the left-hand panel of FIG. 14C), the dilution of the mouse anti-p70s6k antibody was 1:1,000. For the secondary antibody, a 1:5,000 dilution of rabbit anti-mouse conjugated HRP antibody in WESTERNBREEZE® diluent was used.

For the electro-immunoblot, shown in the right-hand panel of FIG. 14C, 3.5 ml of a 1:500 dilution of mouse anti-p70s6k antibody in WESTERNBREEZE® diluent was absorbed on the polyester/polyamide microfiber matrix. The IBLOT™ apparatus was set to program P7 for 5 minutes. The matrix was removed from the apparatus, and 3.5 ml of a 1:5,000 dilution of rabbit anti-mouse conjugated HRP antibody in WESTERNBREEZE® diluent was absorbed on a second polyester/polyamide microfiber matrix, which was placed over the NC membrane. The IBLOT™ apparatus was closed and run at P7 for another 2 minutes. When the program run was complete the membrane was removed, washed three times in WESTERNBREEZE® washing solution and developed using enhanced chemiluminescence as described above.

In FIG. 14D, SW480 cellular lysate was resolved in a NUPAGE® Novex 4-12% Bis-Tris gel. In the conventional immunoblot (shown in the left-hand panel of FIG. 14C), the dilution of the rabbit anti-p53 antibody was 1:5,000. For the secondary antibody, a 1:5,000 dilution of mouse anti-rabbit conjugated HRP antibody in WESTERNBREEZE® diluent was used.

For the electro-immunoblot, shown in the right-hand panel of FIG. 14D, 3.5 ml of a 1:2,500 dilution of mouse anti-p53 antibody in WESTERNBREEZE® diluent was absorbed on the polyester/polyamide microfiber matrix. The IBLOT™ apparatus was set to program P7 for 5 minutes. The matrix was removed from the apparatus, and 3.5 ml of a 1:5,000 dilution of mouse anti-rabbit conjugated HRP antibody in WESTERNBREEZE® diluent was absorbed on a second polyester/polyamide microfiber matrix, which was placed over the NC membrane. The IBLOT™ apparatus was closed and run at P7 for another 2 minutes. When the program run was complete the membrane was removed, washed three times in WESTERNBREEZE® washing solution and developed using enhanced chemiluminescence as described above.

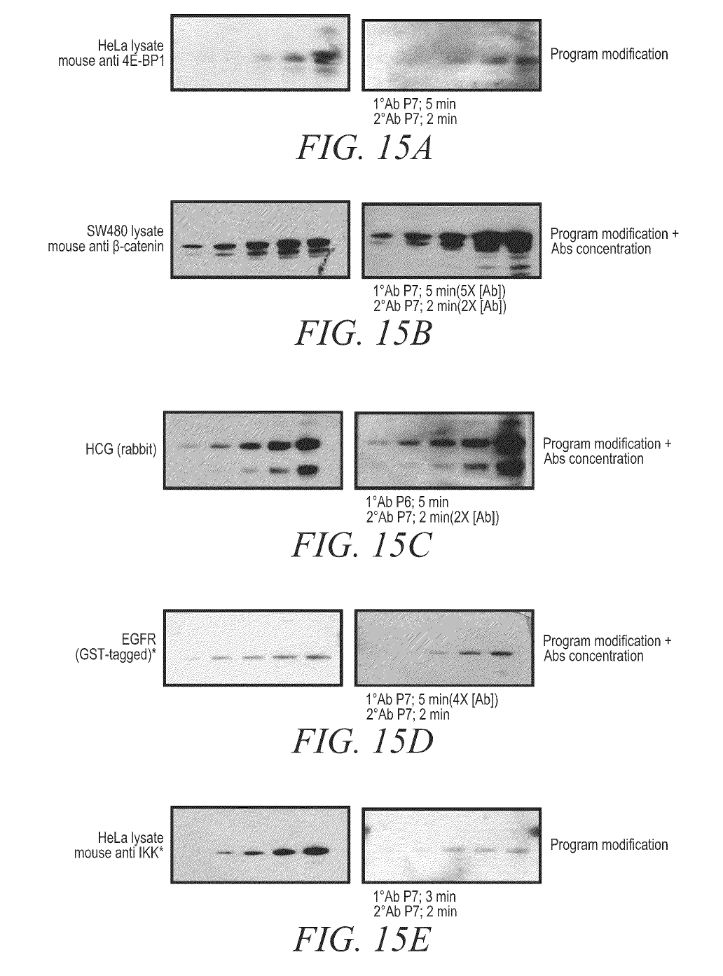

In FIG. 15A, HeLa cellular lysate was resolved in a NUPAGE® Novex 4-12% Bis-Tris gel. In the conventional immunoblot (shown in the left-hand panel of FIG. 15A), the dilution of the mouse anti-4E-BP1 antibody was 1:2,500. For the secondary antibody, a 1:5,000 dilution of rabbit anti-mouse conjugated HRP antibody in WESTERNBREEZE® diluent was used.

For the electro-immunoblot, shown in the right-hand panel of FIG. 15A, 3.5 ml of a 1:2,500 dilution of mouse anti-4E-BP1 antibody in WESTERNBREEZE® diluent was absorbed on the polyester/polyamide microfiber matrix. The IBLOT™ apparatus was set to program P7 for 5 minutes. The matrix was removed from the apparatus, and 3.5 ml of a 1:5,000 dilution of rabbit anti-mouse conjugated HRP antibody in WESTERNBREEZE® diluent was absorbed on a second polyester/polyamide microfiber matrix, which was placed over the NC membrane. The IBLOT™ apparatus was closed and run at P7 for another 2 minutes. When the program run was complete the membrane was removed, washed three times in WESTERNBREEZE® washing solution and developed using enhanced chemiluminescence as described above.

In FIG. 15B, SW480 cellular lysate was resolved in a NUPAGE® Novex 4-12% Bis-Tris gel. In the conventional immunoblot (shown in the left-hand panel of FIG. 15B), the dilution of the mouse anti-β-catenin antibody was 1:2,500. For the secondary antibody, a 1:5,000 dilution of rabbit anti-mouse conjugated HRP antibody in WESTERNBREEZE® diluent was used.

For the electro-immunoblot, shown in the right-hand panel of FIG. 15B, 3.5 ml of a 1:500 dilution of mouse anti-β-catenin antibody in WESTERNBREEZE® diluent was absorbed on the polyester/polyamide microfiber matrix. The IBLOT™ apparatus was set to program P7 for 5 minutes. The matrix was removed from the apparatus, and 3.5 ml of a 1:2,500 dilution of rabbit anti-mouse conjugated HRP antibody in WESTERNBREEZE® diluent was absorbed on a second polyester/polyamide microfiber matrix, which was placed over the NC membrane. The IBLOT™ apparatus was closed and run at P7 for another 2 minutes. When the program run was complete the membrane was removed, washed three times in WESTERNBREEZE® washing solution and developed using enhanced chemiluminescence as described above.

In FIG. 15C, HCG lysate was resolved in a NUPAGE® Novex 4-12% Bis-Tris gel. In the conventional immunoblot (shown in the left-hand panel of FIG. 15C), the dilution of the mouse anti-β-catenin antibody was 1:2,500. For the secondary antibody, a 1:5,000 dilution of rabbit anti-mouse conjugated HRP antibody in WESTERNBREEZE® diluent was used.

For the electro-immunoblot, shown in the right-hand panel of FIG. 15C, 3.5 ml of a 1:1,250 dilution of rabbit anti-HCG antibody in WESTERNBREEZE® diluent was absorbed on the polyester/polyamide microfiber matrix. The IBLOT™ apparatus was set to program P6 for 5 minutes. The matrix was removed from the apparatus, and 3.5 ml of a 1:2,500 dilution of mouse anti-rabbit conjugated HRP antibody in WESTERNBREEZE® diluent was absorbed on a second polyester/polyamide microfiber matrix, which was placed over the NC membrane. The IBLOT™ apparatus was closed and run at P7 for another 2 minutes. When the program run was complete the membrane was removed, washed three times in WESTERNBREEZE® washing solution and developed using enhanced chemiluminescence as described above.

In FIG. 15D, purified EGFR-GST fusion protein was resolved in a NUPAGE® Novex 4-12% Bis-Tris gel. In the conventional immunoblot (shown in the left-hand panel of FIG. 15D), the dilution of the mouse anti-GST antibody was 1:2,500. For the secondary antibody, a 1:5,000 dilution of rabbit anti-mouse conjugated HRP antibody in WESTERNBREEZE® diluent was used.

For the electro-immunoblot, shown in the right-hand panel of FIG. 15D, 3.5 ml of a 1:625 dilution of rabbit anti-GST antibody in WESTERNBREEZE® diluent was absorbed on the polyester/polyamide microfiber matrix. The IBLOT™ apparatus was set to program P7 for 5 minutes. The matrix was removed from the apparatus, and 3.5 ml of a 1:2,500 dilution of mouse anti-rabbit conjugated HRP antibody in WESTERNBREEZE® diluent was absorbed on a second polyester/polyamide microfiber matrix, which was placed over the NC membrane. The IBLOT™ apparatus was closed and run at P7 for another 2 minutes. When the program run was complete the membrane was removed, washed three times in WESTERNBREEZE® washing solution and developed using enhanced chemiluminescence as described above.

In FIG. 15E, HeLa cell lysate was resolved in a NUPAGE® Novex 4-12% Bis-Tris gel. In the conventional immunoblot (shown in the left-hand panel of FIG. 15E), the dilution of the mouse anti-IKKα antibody was 1:2,500. For the secondary antibody, a 1:5,000 dilution of rabbit anti-mouse conjugated HRP antibody in WESTERNBREEZE® diluent was used.

For the electro-immunoblot, shown in the right-hand panel of FIG. 15E, 3.5 ml of a 1:2,500 dilution of mouse anti-IKKα antibody in WESTERNBREEZE® diluent was absorbed on the polyester/polyamide microfiber matrix. The IBLOT™ apparatus was set to program P7 for 3 minutes. The matrix was removed from the apparatus, and 3.5 ml of a 1:2,500 dilution of rabbit anti-mouse conjugated HRP antibody in WESTERNBREEZE® diluent was absorbed on a second polyester/polyamide microfiber matrix, which was placed over the NC membrane. The IBLOT™ apparatus was closed and run at P7 for another 2 minutes. When the program run was complete the membrane was removed, washed three times in WESTERNBREEZE® washing solution and developed using enhanced chemiluminescence as described above.

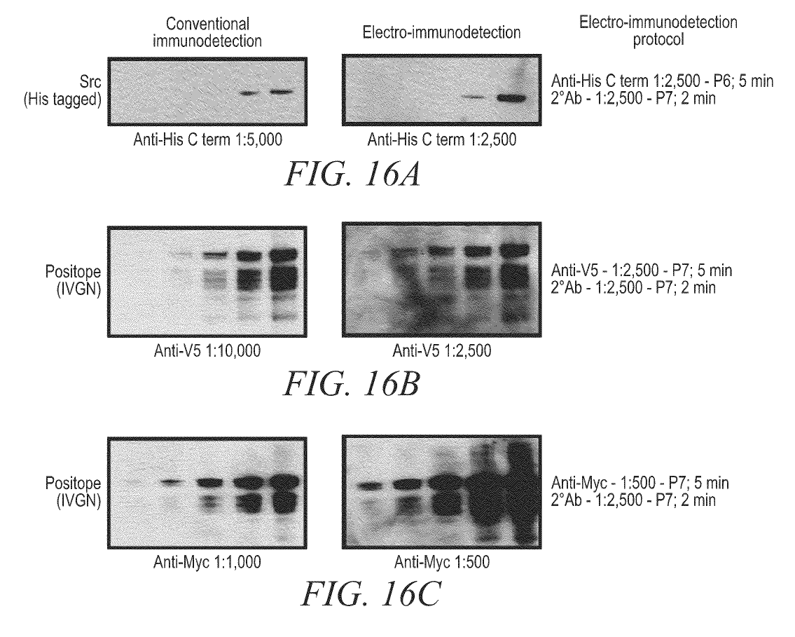

In FIG. 16A, HeLa cell lysate expressing HIS-tagged Src was resolved in a NUPAGE® Novex 4-12% Bis-Tris gel. In the conventional immunoblot (shown in the left-hand panel of FIG. 16A), the dilution of the mouse anti-HIS antibody was 1:5,000. For the secondary antibody, a 1:5,000 dilution of rabbit anti-mouse conjugated HRP antibody in WESTERNBREEZE® diluent was used.

For the electro-immunoblot, shown in the right-hand panel of FIG. 16A, 3.5 ml of a 1:2,500 dilution of mouse anti-HIS antibody in WESTERNBREEZE® diluent was absorbed on the polyester/polyamide microfiber matrix. The IBLOT™ apparatus was set to program P7 for 5 minutes. The matrix was removed from the apparatus, and 3.5 ml of a 1:2,500 dilution of rabbit anti-mouse conjugated HRP antibody in WESTERNBREEZE® diluent was absorbed on a second polyester/polyamide microfiber matrix, which was placed over the NC membrane. The IBLOT™ apparatus was closed and run at P7 for another 2 minutes. When the program run was complete the membrane was removed, washed three times in WESTERNBREEZE® washing solution and developed using enhanced chemiluminescence as described above.

In FIG. 16B, POSITOPE™ control protein (Invitrogen Corp, Carlsbad, Calif.) was resolved in a NUPAGE® Novex 4-12% Bis-Tris gel. In the conventional immunoblot (shown in the left-hand panel of FIG. 16B), the dilution of the mouse anti-V5 antibody was 1:10,000. For the secondary antibody, a 1:5,000 dilution of rabbit anti-mouse conjugated HRP antibody in WESTERNBREEZE® diluent was used.

For the electro-immunoblot, shown in the right-hand panel of FIG. 16B, 3.5 ml of a 1:2,500 dilution of mouse anti-V5 antibody in WESTERNBREEZE® diluent was absorbed on the polyester/polyamide microfiber matrix. The IBLOT™ apparatus was set to program P7 for 5 minutes. The matrix was removed from the apparatus, and 3.5 ml of a 1:2,500 dilution of rabbit anti-mouse conjugated HRP antibody in WESTERNBREEZE® diluent was absorbed on a second polyester/polyamide microfiber matrix, which was placed over the NC membrane. The IBLOT™ apparatus was closed and run at P7 for another 2 minutes. When the program run was complete the membrane was removed, washed three times in WESTERNBREEZE® washing solution and developed using enhanced chemiluminescence as described above.

In FIG. 16C, POSITOPE™ control protein (Invitrogen Corp, Carlsbad, Calif.) was resolved in a NUPAGE® Novex 4-12% Bis-Tris gel. In the conventional immunoblot (shown in the left-hand panel of FIG. 16B), the dilution of the mouse anti-MYC antibody was 1:10,000. For the secondary antibody, a 1:5,000 dilution of rabbit anti-mouse conjugated HRP antibody in WESTERNBREEZE® diluent was used.

For the electro-immunoblot, shown in the right-hand panel of FIG. 16C, 3.5 ml of a 1:2,500 dilution of mouse anti-MYC antibody in WESTERNBREEZE® diluent was absorbed on the polyester/polyamide microfiber matrix. The IBLOT™ apparatus was set to program P7 for 5 minutes. The matrix was removed from the apparatus, and 3.5 ml of a 1:2,500 dilution of rabbit anti-mouse conjugated HRP antibody in WESTERNBREEZE® diluent was absorbed on a second polyester/polyamide microfiber matrix, which was placed over the NC membrane. The IBLOT™ apparatus was closed and run at P7 for another 2 minutes. When the program run was complete the membrane was removed, washed three times in WESTERNBREEZE® washing solution and developed using enhanced chemiluminescence as described above.

### Example 14

### Comparison of Electro-Blotting with an Alternate Commercially Available Immunodetection System

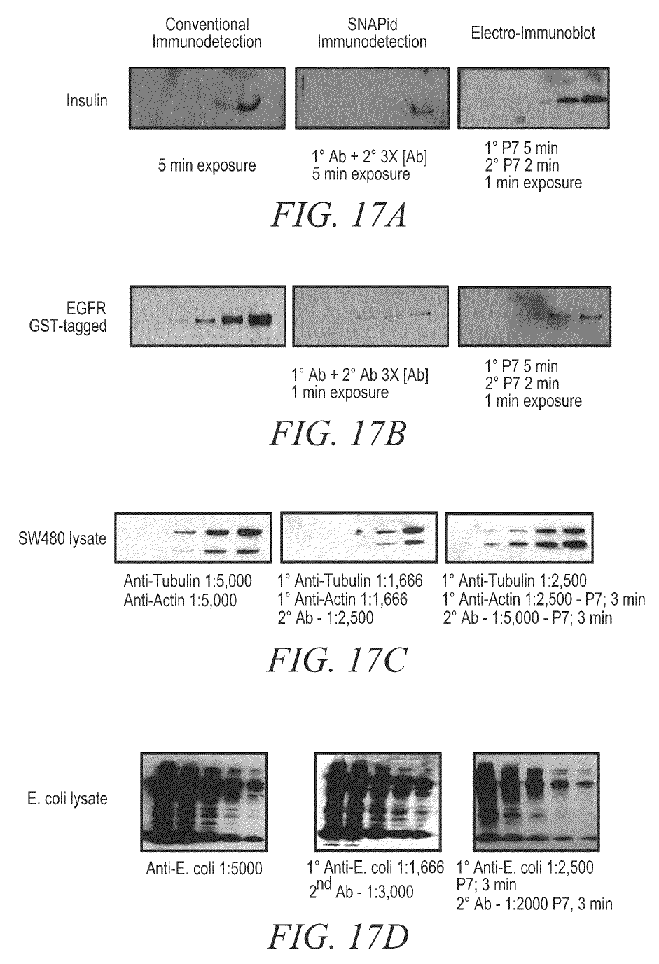

The experiments shown in FIGS. 17A-D were performed to compare the results obtained using three different immunodetection methods, namely; conventional western blot (shown in the left-hand panels of FIGS. 17A-17D), SNAP ID™ Protein Detection System (Millipore Corporation, Billerica, Mass.; shown in the middle panels of FIGS. 17A-17D), and electro-immunoblot (shown the right-hand panels of FIGS. 17A-17D).

In FIG. 17A, purified recombinant insulin was resolved on three separate NUPAGE® Novex 4-12% Bis-Tris gels. The resolved proteins were transferred to NC membranes using the IBLOT™ apparatus as described above.

The control membrane (shown on the left hand panel of FIG. 17A) was subjected to conventional blotting as described above using 1:5000 dilution of rabbit anti-insulin antibody diluted in WESTERNBREEZE™ diluent for 1 hour at room temperature. The antibody solution was discarded, and the membrane washed three times for 5 minutes each in WESTERNBREEZE™ diluent. A 1:5000 dilution of mouse anti-rabbit conjugated HRP antibody in WESTERNBREEZE™ diluent was applied to the membrane for 30 minutes at room temperature. The washing steps were repeated, and the blot was developed using enhanced chemiluminescence (ECL) as described above. The ECL-treated blot was exposed to film for 5 minutes and developed (see left-hand panel).

A second membrane (shown in the middle panel of FIG. 17A) was subjected to blotting using the SNAP ID™ Protein Detection System according to manufacturer's instruction. The dilution of the anti-insulin antibody and the anti-rabbit HRP antibody that was used in this experiment was 1:1650. The blot was developed using enhanced chemiluminescence (ECL) as described above. The ECL-treated blot was exposed to film for 5 minutes and developed (see middle panel).

For the electro-immunoblot, shown in the right-hand panel of FIG. 17A, 3.5 ml of a 1:2,500 dilution of mouse anti-insulin antibody in WESTERNBREEZE® diluent was absorbed on the polyester/polyamide microfiber matrix. The IBLOT™ apparatus was set to program P7 for 5 minutes. The matrix was removed from the apparatus, and 3.5 ml of a 1:2,500 dilution of mouse anti-rabbit conjugated HRP antibody in WESTERNBREEZE® diluent was absorbed on a second polyester/polyamide microfiber matrix, which was placed over the NC membrane. The IBLOT™ apparatus was closed and run at P7 for another 2 minutes. When the program run was complete the membrane was removed, washed three times in WESTERNBREEZE® washing solution and developed using enhanced chemiluminescence as described above. The blot was developed using enhanced chemiluminescence (ECL) as described above. The ECL-treated blot was exposed to film for 1 minutes and developed (see right-hand panel).

In FIG. 17B, purified recombinant GST-tagged EGFR fusion protein was resolved on three separate NUPAGE® Novex 4-12% Bis-Tris gels. The resolved proteins were transferred to NC membranes using the IBLOT™ apparatus as described above.

The control membrane (shown on the left hand panel of FIG. 17B) was subjected to conventional blotting as described above using 1:2,500 dilution of mouse anti-GST antibody diluted in WESTERNBREEZE™ diluent for 1 hour at room temperature. The antibody solution was discarded, and the membrane washed three times for 5 minutes each in WESTERNBREEZE™ diluent. A 1:5000 dilution of rabbit anti-mouse conjugated HRP antibody in WESTERNBREEZE™ diluent was applied to the membrane for 30 minutes at room temperature. The washing steps were repeated, and the blot was developed using enhanced chemiluminescence (ECL) as described above. The ECL-treated blot was exposed to film for 5 minutes and developed (see left-hand panel).

A second membrane (shown in the middle panel of FIG. 17B) was subjected to blotting using the SNAP ID™ Protein Detection System according to manufacturer's instruction. The dilution of the anti-GST antibody and the anti-mouse HRP antibody that was used in this experiment was 1:850 and 1:1,650, respectively. The blot was developed using enhanced chemiluminescence (ECL) as described above. The ECL-treated blot was exposed to film for 1 minute and developed (see middle panel).

For the electro-immunoblot, shown in the right-hand panel of FIG. 17B, 3.5 ml of a 1:2,500 dilution of mouse anti-GST antibody in WESTERNBREEZE® diluent was absorbed on the polyester/polyamide microfiber matrix. The IBLOT™ apparatus was set to program P7 for 6 minutes. The matrix was removed from the apparatus, and 3.5 ml of a 1:2,500 dilution of rabbit anti-mouse conjugated HRP antibody in WESTERNBREEZE® diluent was absorbed on a second polyester/polyamide microfiber matrix, which was placed over the NC membrane. The IBLOT™ apparatus was closed and run at P7 for another 2 minutes. When the program run was complete the membrane was removed, washed three times in WESTERNBREEZE® washing solution and developed using enhanced chemiluminescence as described above. The blot was developed using enhanced chemiluminescence (ECL) as described above. The ECL-treated blot was exposed to film for 1 minutes and developed (see right-hand panel).

In FIG. 17C, SW480 cellular lysate was resolved on three separate NUPAGE® Novex 4-12% Bis-Tris gels. The resolved proteins were transferred to NC membranes using the IBLOT™ apparatus as described above.

The control membrane (shown on the left hand panel of FIG. 17C) was subjected to conventional blotting as described above using 1:5,000 dilution of mouse anti-tubulin antibody and a 1:5,000 dilution of mouse anti-actin antibody diluted in WESTERNBREEZE™ diluent for 1 hour at room temperature. The antibody solution was discarded, and the membrane washed three times for 5 minutes each in WESTERNBREEZE™ diluent. A 1:5000 dilution of rabbit anti-mouse conjugated HRP antibody in WESTERNBREEZE™ diluent was applied to the membrane for 30 minutes at room temperature. The washing steps were repeated, and the blot was developed using enhanced chemiluminescence (ECL) as described above. The ECL-treated blot was exposed to film for 1 minute and developed (see left-hand panel).

A second membrane (shown in the middle panel of FIG. 17C) was subjected to blotting using the SNAP ID™ Protein Detection System according to manufacturer's instruction. The dilution of the anti-tubulin antibody and anti-actin antibody was 1:1,650, and the dilution of the anti-mouse HRP antibody that was used in this experiment was 1:2,500. The blot was developed using enhanced chemiluminescence (ECL) as described above. The ECL-treated blot was exposed to film for 1 minute and developed (see middle panel).

For the electro-immunoblot, shown in the right-hand panel of FIG. 17C, 3.5 ml of a 1:2,500 dilution of mouse anti-tubulin and a 1:2,500 dilution of mouse anti-actin antibody in WESTERNBREEZE® diluent was absorbed on the polyester/polyamide microfiber matrix. The IBLOT™ apparatus was set to program P7 for 3 minutes. The matrix was removed from the apparatus, and 3.5 ml of a 1:5,000 dilution of rabbit anti-mouse conjugated HRP antibody in WESTERNBREEZE® diluent was absorbed on a second polyester/polyamide microfiber matrix, which was placed over the NC membrane. The IBLOT™ apparatus was closed and run at P7 for another 3 minutes. When the program run was complete the membrane was removed, washed three times in WESTERNBREEZE® washing solution and developed using enhanced chemiluminescence as described above. The blot was developed using enhanced chemiluminescence (ECL) as described above. The ECL-treated blot was exposed to film for 1 minutes and developed (see right-hand panel).

In FIG. 17D, E. coli lysate was resolved on three separate NUPAGE® Novex 4-12% Bis-Tris gels. The resolved proteins were transferred to NC membranes using the IBLOT™ apparatus as described above.

The control membrane (shown on the left hand panel of FIG. 17D) was subjected to conventional blotting as described above using 1:5,000 dilution of rabbit anti-E. coli antibody diluted in WESTERNBREEZE™ diluent for 1 hour at room temperature. The antibody solution was discarded, and the membrane washed three times for 5 minutes each in WESTERNBREEZE™ diluent. A 1:5000 dilution of mouse anti-rabbit conjugated HRP antibody in WESTERNBREEZE™ diluent was applied to the membrane for 30 minutes at room temperature. The washing steps were repeated, and the blot was developed using enhanced chemiluminescence (ECL) as described above. The ECL-treated blot was exposed to film for 1 minute and developed (see left-hand panel).

A second membrane (shown in the middle panel of FIG. 17D) was subjected to blotting using the SNAP ID™ Protein Detection System according to manufacturer's instruction. The dilution of the anti-E. coli antibody was 1:1,650, and the dilution of the anti-rabbit HRP antibody that was used in this experiment was 1:3,000. The blot was developed using enhanced chemiluminescence (ECL) as described above. The ECL-treated blot was exposed to film for 1 minute and developed (see middle panel).

For the electro-immunoblot, shown in the right-hand panel of FIG. 17D, 3.5 ml of a 1:2,500 dilution of mouse anti-E. coli antibody in WESTERNBREEZE® diluent was absorbed on the polyester/polyamide microfiber matrix. The IBLOT™ apparatus was set to program P7 for 3 minutes. The matrix was removed from the apparatus, and 3.5 ml of a 1:5,000 dilution of mouse anti-rabbit conjugated HRP antibody in WESTERNBREEZE® diluent was absorbed on a second polyester/polyamide microfiber matrix, which was placed over the NC membrane. The IBLOT™ apparatus was closed and run at P7 for another 3 minutes. When the program run was complete the membrane was removed, washed three times in WESTERNBREEZE® washing solution and developed using enhanced chemiluminescence as described above. The blot was developed using enhanced chemiluminescence (ECL) as described above. The ECL-treated blot was exposed to film for 1 minute and developed (see right-hand panel).

### Electro-Blotting to Detect Nucleic Acids

### Example 15

In this example, the electro-blotting systems and methods described above were adapted for use in performing a nucleic acid blotting experiment (i.e., a Southern blot).

Lambda DNA (12.5-0.6 ng/well) samples were run on 3 identical 0.8% agarose slab gels for 2 hours at 100 V. The gels were transferred to nylon membranes using the IBLOT™ device (P8 7 minutes) using the IBLOT™ NAT stacks (Invitrogen, Carlsbad, Calif.). Following transfer of the resolved nucleic acids to the nylon membranes, the immobilized nucleic acid was denatured by immersing the nylon membrane in an aqueous solution of 0.4 N NaOH for 10 minutes. The nylon membranes were then irradiated with UV light to crosslinked the nucleic acid to the membrane.

The treated membranes were then subjected to a pre-hybridization treatment for 2 hours at 55° C. in hyrbridization buffer prepared according to instructions provided in the AMERSHAM ALKPHOS DIRECT™ Labeling and Detection kit (GE Healthcare, Uppsala, Sweden).

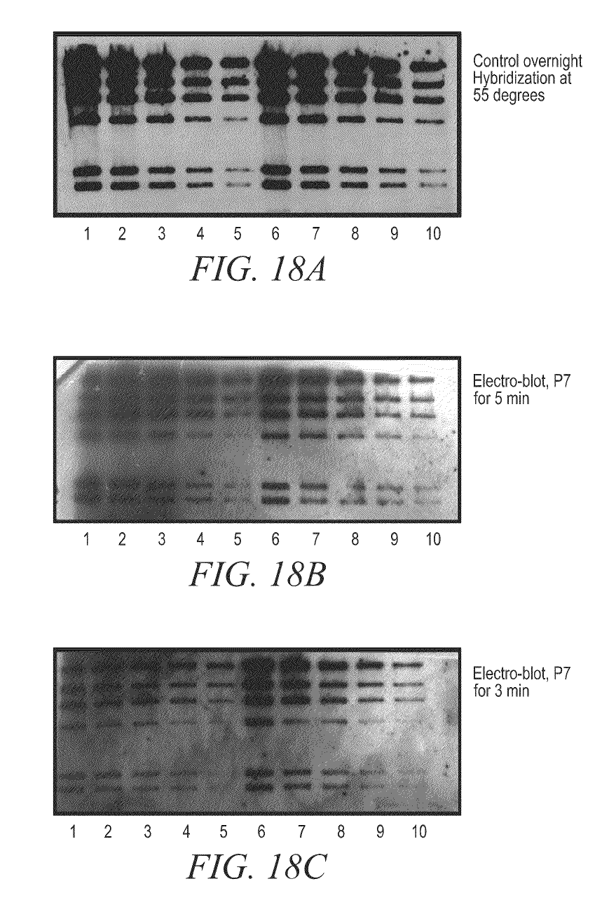

One of the membranes (control membrane depicted in FIG. 18A) was incubated overnight at 55° C. with rotation with 12.5 ml of pre-hybridization buffer containing 62.5 ng of DNA probe (Lambda DNA labeled with alkaline phosphatase according to manufacturer instructions).

The remaining two test membranes were each placed over an iBlot bottom stack and 5 ml of pre-hybridization buffer containing 187 ng of the prepared DNA probe was absorbed onto the matrices. A top stack was placed over each of the probe-soaked matrices and the assembly was inserted into the IBLOT™ device according to manufacturer instructions. The assembly was run on P7 was for either 5 minutes (shown in FIG. 18B) or 3 minutes (shown in FIG. 18C). After the hybridization (either conventional or the electro-blotting method), the membranes were washed according to instructions of the AMERSHAM ALKPHOS DIRECT™ Labeling and Detection kit and then chemiluminescently developed using CDP-STAR® Reagent (NEB, Ipswich, Mass.) as substrate. All three membranes were exposed to X-ray film for 1 hour, and the film was developed. Results are shown in FIG. 18A-C

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-89. (canceled)

90. A kit for performing electro-immunoblotting, said kit comprising in at least a first suitable container:
an anodic assembly;
a cathodic assembly comprising; and
a first carrier matrix.

91. The kit according to claim 90, wherein the anodic assembly comprises an anodic gel matrix body.

92. The kit according to claim 90, wherein the anodic assembly comprises an anode.

93. The kit according to claim 90, wherein the cathodic assembly comprises a cathodic gel matrix.

94. The kit according to claim 90, wherein the anodic assembly comprises a cathode.

95. The kit according to claim 90, further comprising an optional second carrier matrix.

96. The kit according to claim 90, further comprising an anode coupled to the anodic gel matrix body.

97. The kit according to claim 90, further comprising a cathode coupled to the cathodic gel matrix body.

98. The kit according to claim 90, wherein the anodic assembly and the cathodic assembly are packaged separately.

99. The kit according to claim 90, further comprising at least one tray sized to accept the anodic assembly or the cathodic assembly.

100. The kit according to claim 90, further comprising at least one aqueous buffer.

101. The kit according to claim 100, wherein the aqueous buffer is a blocking buffer.

102. The kit according to claim 100, wherein the aqueous buffer comprises at least one blocking reagent.

103. The kit according to claim 100, wherein the aqueous buffer comprises at least one synthetic blocking reagent.

104. The kit according to claim 10303, wherein the synthetic blocking reagent comprises from about 1% to about 50% of a synthetic blocking reagent.

105. The kit according to claim 1022, wherein the blocking reagent comprises a proteinaceous or hybridization composition.

106. The kit according to claim 1022, wherein the blocking reagent comprises at least one protein composition selected from gelatin, non-fat milk, casein, BSA, CAS-Block, soy protein, goat immunoglobulin, rabbit, immunoglobulin, mouse immunoglobulin, rat immunoglobulin, horse immunoglobulin, human immunoglobulin, pig immunoglobulin, chicken immunoglobulin, synthetic peptides, rice proteins, whey proteins, fish proteins algae proteins or any combinations thereof.

107. The kit according to claim 10606, wherein the blocking reagent comprises between 0.25 wt. % and 10 wt. % of at least one protein composition selected from gelatin, non-fat milk, casein, BSA, CAS-Block, soy protein, a synthetic blocking reagent, goat immunoglobulin, rabbit, immunoglobulin, mouse immunoglobulin, rat immunoglobulin, horse immunoglobulin, human immunoglobulin, pig immunoglobulin, chicken immunoglobulin, synthetic peptides, rice proteins, whey proteins, fish proteins algae proteins or any combinations thereof.

108. The kit according to claim 90, further comprising an aqueous wash buffer.

109. The kit according to claim 90, wherein the anodic assembly is electrically coupleable to the cathodic assembly.

110. The kit according to claim 90, further comprising one or more primary antibodies.

111. The kit according to claim 90, further comprising one or more secondary antibodies.

112. The kit according to claim 90, further comprising one or more sponges.

113. The kit according to claim 90, further comprising one or more sheets of filter paper.

114. A method for performing electro-immunoblotting, said method comprising:
providing an anodic assembly comprising an anode and a source of ions for electrophoresis;
providing a cathodic assembly comprising a cathode and a source of ions for electrophoresis;
providing a first carrier matrix comprising a proteinaceous or hybridization composition absorbed thereon;
providing a protein blotting membrane comprising a protein sample coupled to a surface thereof;
positioning the protein blotting membrane and the first carrier matrix between the anodic assembly and the cathodic assembly such that the first carrier matrix is proximal to the cathode assembly, the protein blotting membrane is proximal to the anodic assembly, and the surface of the protein blotting membrane having the protein sample coupled thereto is substantially juxtaposed with a surface of the first carrier matrix; and
applying a voltage between the anodic assembly and the cathodic assembly.

115. The method according to claim 113, wherein the anodic assembly comprises an anodic gel matrix body.

116. The method according to claim 113, wherein the cathodic assembly comprises a cathodic gel matrix body.

117. The method according to claim 113, wherein the proteinaceous or hybridization composition comprises a blocking reagent.

118. The method according to claim 113, wherein the proteinaceous or hybridization composition comprises a primary antibody.

119. The method according to claim 113, wherein the proteinaceous or hybridization composition comprises a secondary antibody.

120. The method according to claim 113, wherein the proteinaceous or hybridization composition comprises a nucleic acid probe.

121. The method according to claim 113, wherein the proteinaceous or hybridization composition is absorbed on the first carrier matrix prior to the positioning step.

122. The method according to claim 121, wherein the absorbing step comprises contacting the first carrier matrix with an aqueous solution comprising the proteinaceous or hybridization composition.

123. The method according to claim 114, wherein the voltage is up to about 50V.

124. The method according to claim 114, wherein the voltage is up to about 25V.

125. The method according to claim 114, wherein the voltage is up to about 15V.

126. The method according to claim 114, wherein the voltage is up to about 5V.

127. The method according to claim 114, wherein the voltage is applied for up to about 15 minutes.

128. The method according to claim 114, wherein the voltage is applied for up to about 10 minutes.

129. The method according to claim 114, wherein the voltage is applied for up to about 5 minutes.

130. The method according to claim 114, wherein the voltage is applied for between about 1 to about 5 minutes.

131. The method according to claim 114, wherein the voltage is applied for between about 1 to about 3 minutes.

132. The method according to claim 114, wherein the voltage is applied for about 3 minutes.

133. The method according to claim 114, wherein after the voltage is applied between the anodic assembly and the cathodic assembly, the first carrier matrix is replaced with a second carrier matrix, said second carrier matrix comprising a proteinaceous or hybridization composition absorbed thereon.

134. The method according to claim 133, wherein a voltage is applied between the anodic assembly and the cathodic assembly after the first carrier matrix is replaced with the second carrier matrix.

135. The method according to claim 113, further comprising subjecting the protein blotting membrane to one or more washing steps.

136. The method according to claim 135, wherein the protein blotting membrane is subjected to at least three washes.

137. The method according to claim 135, wherein at least one washing step is about 1-5 minutes.

138. The method according to claim 113, further comprising subjecting the protein blotting membrane to a detection step.

139. The method according to claim 138, wherein the detection step comprises a chemiluminescent detection step.

140. The method according to claim 138, wherein the detection step comprises a calorimetric detection step.

141. The method according to claim 138, wherein the detection step comprises a fluorescent detection step.

142. A system for performing electro-immunoblotting, said system comprising:
an anodic assembly;
a cathodic assembly; and
a first carrier matrix, wherein the first carrier matrix comprises polyester/polyamide microfibers.
